SEQUENCE LISTING (66 sequences)

SEQ ID NO: 1 - protein, 28 aa, Homo sapiens
PEGGGLMDSDETGFEAHXPGILAPQPNH

SEQ ID NO: 2 - protein, 28 aa, Homo sapiens
PEGGGLMDSDETGFEAHXPGKLAPQPNH

SEQ ID NO: 3 - protein, 24 aa, Homo sapiens
PEGGGLMRWQEMGYIFYPRKLRNH

SEQ ID NO: 4 - protein, 11 aa, Homo sapiens
PEGGGLKPVNH

SEQ ID NO: 5 - protein, 39 aa, Homo sapiens
PEGGGLXDHFRWKNHACNLEASPHISDPHEARGTRPLYS

SEQ ID NO: 6 - protein, 35 aa, Homo sapiens
PEGGGLNLECYCLOASPHISDPHEARGTRPLYSNH

SEQ ID NO: 7 - protein, 35 aa, Homo sapiens; identical to SEQ ID NO: 6

SEQ ID NO: 8 - protein, 23 aa, Homo sapiens
PEGGGLGEPKPGKPADDAGLVNH

SEQ ID NO: 9 - protein, 60 aa, Homo sapiens
PEGGGLACETYLSDKPDMAEIEKFDKSKLKAHXKTETQEKNPLPSKETIEQEKQAGESNH

SEQ ID NO: 10 - protein, 54 aa, Homo sapiens
PEGGGLSDKPDMAEIEKFDKSKLKAHXKTETQEKNPLPSKETIEQEKQAGESNH

SEQ ID NO: 11 - protein, 17 aa, Homo sapiens
PEGGGLGHKNHCUXHAC

SEQ ID NO: 12 - protein, 31 aa, Homo sapiens
PEGGGLGHKAHXACSSSPSKHCGNHCUXHAC

SEQ ID NO: 13 - protein, 15 aa, Homo sapiens
PEGGGLTKPRPGPNH

SEQ ID NO: 14 - protein, 33 aa, Homo sapiens
PEGGGLHWAWFKAHXYLRIVQCRSVEGSCGFNH

SEQ ID NO: 15 - protein, 17 aa, Homo sapiens
PEGGGLXAKSQGGSNNH

SEQ ID NO: 16 - protein, 20 aa, Homo sapiens
PEGGGLKPYRAKSQGGSNNH

SEQ ID NO: 17 - protein, 36 aa, Homo sapiens
PEGGGLHSDAVFTDNYTRLRKQMAVKKYLNSILNNH

SEQ ID NO: 18 - protein, 39 aa, Homo sapiens
PEGGGLSDAAVDTSSEITTAHXKDLKEKKEVVEEAENNH

SEQ ID NO: 19 - protein, 12 aa, Homo sapiens
PEGGGLAEDGNH

SEQ ID NO: 20 - protein, 24 aa, Homo sapiens
PEGGGLDESHGTAVMWIFLKYPNH

SEQ ID NO: 21 - protein, 16 aa, Homo sapiens
PEGGGLRXDIMEKFNH

SEQ ID NO: 22 - protein, 19 aa, Homo sapiens
PEGGGLDRTYRDIMEKFNH

SEQ ID NO: 23 - protein, 29 aa, Homo sapiens
PEGGGLLRRRLSDANFAHXRRRRRRRRNH

SEQ ID NO: 24 - protein, 192 aa, Homo sapiens
PEGGGLDLEUDTHRDLEUDARGDLYSDGLUDPRODALADSERDGLUDILEDALADGLNDSERDILEDLEUDGLUDALADTYRDSERDGLNDASNDGLYDTRPDALADASNDARGDARGDSERDGLYDGLYDLYSDARGDPRODPRODPRODARGDARGDARGDGLNDARGDARGDLYSDLYSDARGDGLYNH

SEQ ID NO: 25 - protein, 196 aa, Homo sapiens
PEGGGLDLEUDTHRDLEUDARGDLYSDGLUDPRODALADSERDGLUISOLEUDALADGLNDSERISOLEUDLEUDGLUDALADTYRDSERDGLNDASNDGLYDTRPDALADASNDARGDARGDSERDGLYDGLYDLYSDARGDPRODLYSDPRODARGDARGDARGDGLNDARGDARGDLYSDLYSDARGDGLYNH

SEQ ID NO: 26 - protein, 45 aa, Homo sapiens
PEGGGLLLGDFFRKSKEKIGKEFKRIVQRIKDFLRNLVPRTESNH

SEQ ID NO: 27 - protein, 15 aa, Homo sapiens
PEGGGLMEHFPGPNH

SEQ ID NO: 28 - protein, 32 aa, Homo sapiens
PEGGGLTHADILPTNVGSRGARAGLLGLKDNH

SEQ ID NO: 29 - protein, 18 aa, Homo sapiens
PEGGGLYNWNSFGLRFNH

SEQ ID NO: 30 - protein, 16 aa, Homo sapiens
PEGGGLGGVLVQPGNH

SEQ ID NO: 31 - protein, 38 aa, Homo sapiens
PEGGGLFQGTFPDGFLWAVGSAAYQTEGGWQQHGKGNH

SEQ ID NO: 32 - protein, 46 aa, Homo sapiens
PEGGGLIPGPTFLDPHAHXGPYTHDCPVKAHXSGPIMLDEVRGDNH

SEQ ID NO: 33 - protein, 19 aa, Homo sapiens
PEGGGLIQLLSSELLDENH

SEQ ID NO: 34 - protein, 19 aa, Homo sapiens
PEGGGLXEQLERALNSSNH

SEQ ID NO: 35 - protein, 58 aa, Homo sapiens
PEGGGLYDADAIFTQSYRKVLAQLSARKLLQDILSRAHXAIBHISDNALDPHELYSNH

SEQ ID NO: 36 - protein, 37 aa, Homo sapiens
PEGGGLYADAIFTNSYRKVLGQLSARKLLQDIMSRNH

SEQ ID NO: 37 - protein, 55 aa, Homo sapiens
PEGGGLYADAIFTNSYRKVLGQLSARKLAHXLQDIMSRQQGESNQERGARARLNH

SEQ ID NO: 38 - protein, 13 aa, Homo sapiens
PEGGGLYGGFMNH

SEQ ID NO: 39 - protein, 31 aa, Homo sapiens
PEGGGLAGYKPDEGKRGDACEGDSGGPFVNH

SEQ ID NO: 40 - protein, 68 aa, Homo sapiens
PEGGGLHAEGTFTSDVSSYLEGQAAKPEGPEGGLUCARBOXYHEPTADECANOYLEFIAWLVRGRGNH

SEQ ID NO: 41 - protein, 70 aa, Homo sapiens
PEGGGLHAIBEGTFTSDVSSYLEGQAAKPEGPEGGLUCARBOXYHEPTADECANOYLEFIAWLVRGRGNH

SEQ ID NO: 42 - protein, 59 aa, Homo sapiens
PEGGGLYAGTFTSDYSILDKIAQEGEDIACIDEAEEAKAFAQWLIAAGGPSGGPPPSNH

SEQ ID NO: 43 - protein, 62 aa, Homo sapiens
PEGGGLYAIBEGTFTSDYSIAIBLDKIAQDIACIDEAEEAKAFVQWLIAGGPSSGAPPPSNH

SEQ ID NO: 44 - protein, 50 aa, Homo sapiens
PEGGGLYAQGTFTSDYSILLDKKAHXAQAAFIEYLLEGGPSSGAPPPSNH

SEQ ID NO: 45 - protein, 72 aa, Homo sapiens
PEGGGLYAIBQGTFTSDYSIMELEULDKKAEEAGLUCDIACIDAHXAQAIBAFIEYLLEGGPSSGAPPPSNH

SEQ ID NO: 46 - protein, 30 aa, Homo sapiens
PEGGGLNLEASPHISDPHEARGTRPLYSNH

SEQ ID NO: 47 - protein, 28 aa, Homo sapiens
PEGGGLWPYLFYVQAHXLQDMLWQLDNH

SEQ ID NO: 48 - protein, 28 aa, Homo sapiens
PEGGGLKPYLFYVQAHXLQDMLWQLDNH

SEQ ID NO: 49 - protein, 35 aa, Homo sapiens
PEGGRKKRRQRRRAHXRGDMKLVFLVLLFLGALNH

SEQ ID NO: 50 - protein, 35 aa, Homo sapiens
PEGGRKKRRQRRRAHXRGDMKLKFLKLLKLGALNH

SEQ ID NO: 51 - protein, 41 aa, Homo sapiens
PEGGGLACYCRIPACIAGERRYRGDGTCIYQGRLWAFCCNH

SEQ ID NO: 52 - protein, 34 aa, Homo sapiens
PEGGGLGIGKFLHSAKRGDKFGKAFVGEIMNSNH

SEQ ID NO: 53 - protein, 36 aa, Homo sapiens
PEGGGLGIGAVLKVLTTGLPALISWIKRKRQRGDNH

SEQ ID NO: 54 - protein, 36 aa, Homo sapiens
PEGGGLGIGAVLKVLTTGLPKLISWIKRKRQRGDNH

SEQ ID NO: 55 - protein, 39 aa, Homo sapiens
PEGGRKKRRQRRRAHXTTYADFIASGRTGRRNAIRGDNH

SEQ ID NO: 56 - protein, 54 aa, Homo sapiens
PEGGRKKRRQRRRAHXPPLSQETFSDLWKLLOKKWKMRRNQFWVKVQRGRGDNH

SEQ ID NO: 57 - protein, 53 aa, Homo sapiens
PEGGRKKRRQRRRAHXPPLSQETFSDLWKLLKKWKMRRNQFWVKVQRGRGDNH

SEQ ID NO: 58 - protein, 48 aa, Homo sapiens
PEGGRKKRRQRRRAHXETFSDLWKLLOKKWKMRRNQFWVKVRGRGDNH

SEQ ID NO: 59 - protein, 48 aa, Homo sapiens
PEGGRKKRRQRRRAHXETFSDLWKLLKKWKMRRNQFWVKVQRGRGDNH

SEQ ID NO: 60 - protein, 33 aa, Homo sapiens
PEGGRKKRRQRRRAHXDWWPLAFEALLRRGDNH

SEQ ID NO: 61 - protein, 38 aa, Homo sapiens
PEGGGLKCHNOAKSQGGSNOHRGDDWWPLAFEALLRNH

SEQ ID NO: 62 - protein, 34 aa, Homo sapiens
PEGGRKKRRQRRRAHXQRLGHQWAVGHLMRGDNH

SEQ ID NO: 63 - protein, 34 aa, Homo sapiens
PEGGRKKRRQRRRAHXQRLGNQWAVGHLMRGDNH

SEQ ID NO: 64 - protein, 33 aa, Homo sapiens
PEGGGLGRKKRRQRRRAHXSANSNPAMAPRENH

SEQ ID NO: 65 - protein, 31 aa, Homo sapiens
PEGGRKKRRQRRRAHXYNWNSFGLRFRGDNH

SEQ ID NO: 66 - protein, 48 aa, Homo sapiens
PEGGRKKRRQRRRAHXMRKEFHNVLSSDQLLTDKRPARDYNRKRGDNH

(19) United States
(12) Patent Application Publication
(10) Pub. No.: US 2025/0312463 A1
(43) Pub. Date: Oct. 9, 2025

(54) AUGMENTED PEPTIDE COMPOSITIONS AND METHODS

(71) Applicant: Neo7Bioscience, Inc., Dallas, TX (US)

(72) Inventor: John A. Catanzaro, Rockwall, TX (US)

(73) Assignee: Neo7Bioscience, Inc., Dallas, TX (US)

(21) Appl. No.: 19/169,474

(22) Filed: Apr. 3, 2025

Related U.S. Application Data
(60) Provisional application No. 63/573,778, filed on Apr. 3, 2024.

(51) Int. Cl. A61K 47/60

(52) U.S. Cl. CPC A61K 47/60

(57) ABSTRACT

Compositions and methods involving augmented peptides are described herein. In an embodiment, a composition including an augmented peptide may include a therapeutic peptide moiety conjugated to a polyethylene glycol (PEG) moiety, and at least a linking agent, wherein the at least a linking agent is configured to link at least two molecular entities.

### CROSS-REFERENCE TO RELATED APPLICATIONS

This application claims the benefit of priority of U.S. Provisional Patent Application Ser. No. 63/573,778, filed on Apr. 3, 2024, and titled “AUGMENTED PEPTIDE COMPOSITIONS AND METHODS,” which is incorporated by reference herein in its entirety.

### REFERENCE TO SEQUENCE LISTING

This specification includes a sequence listing submitted herewith, which includes the file entitled 1399-002USU1_Sequence_Listing.xml having the following size 59,305 bytes, which was created Mar. 18, 2025, the contents of which are incorporated by reference herein.

### FIELD OF THE INVENTION

The present invention generally relates to the field of therapeutic compositions. In particular, the present invention is directed to augmented peptide compositions and methods.

### SUMMARY OF THE DISCLOSURE

In an aspect, a composition including an augmented peptide may include a therapeutic peptide moiety conjugated to a polyethylene glycol (PEG) moiety, wherein the therapeutic peptide moiety is identified as a function of an integrated multiomic and immunophenotypic data associated with an individual using multiomic and immune modeling and at least a linking agent, wherein the linking agent is configured to link at least two molecular entities.

In another aspect, a method of treating a disease or condition in a subject using an augmented peptide composition may include receiving an integrated multiomic and immunophenotypic data associated with an individual, identifying a therapeutic peptide moiety as a function of the integrated multiomic and immunophenotypic data associated with an individual using multiomic and immune modeling, conjugating the therapeutic peptide moiety to a PEG moiety to form and augmented peptide, conjugating at least a linker agent to the augmented peptide to link at least two molecular entities, and administering an effective amount of the augmented peptide composition to the individual.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an illustration of an exemplary augmented peptide composition;

FIG. 2 is an illustration describing hallmarks of cancer; and

FIG. 3 is a flow diagram of an exemplary method of treating a disease or condition in a subject using an augmented peptide composition; and

FIG. 4 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to compositions and methods related to augmented peptides.

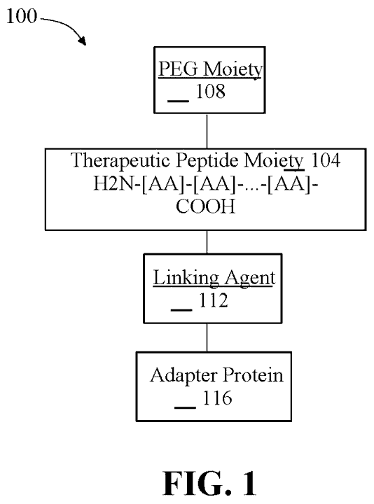

Referring now to FIG. 1, an augmented peptide composition 100 is illustrated. In some embodiments, one or more steps of one or more processes described herein may be performed using a computing device. A computing device may include a processor. Processor may include, without limitation, any processor described in this disclosure. Processor may be included in computing device. Computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device.

Still referring to FIG. 1, in some embodiments, computing device may include at least a processor and a memory communicatively connected to the at least a processor, the memory containing instructions configuring the at least a processor to perform one or more processes described herein. Computing device may include processor and/or memory. Computing device may be configured to perform one or more processes described herein.

With continued reference to FIG. 1, in an embodiment, computing device may include but is not limited to, for example, a computing device or cluster of computing devices in a first location and a second computing device or cluster of computing devices in a second location. Computing device may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for distribution of tasks or memory between computing devices. Computing device may be implemented, as a non-limiting example, using a “shared nothing” architecture.

In further reference to FIG. 1, in an embodiment, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Unless otherwise indicated, the practice of the present invention will employ conventional techniques of cell culturing, molecular biology (including recombinant techniques), microbiology, cell biology, biochemistry, and immunology.

As used herein, the singular forms “a,” “an,” and “the” designate both the singular and the plural, unless expressly stated to designate the singular only.

As used herein, the terms “administer,” “administering,” “administration,” or the like refer to the placement of a composition into a subject by any method. A composition described herein may be administered to a subject by any one of a variety of manners or a combination of varieties of manners. For example, a composition may be administered orally, nasally, intraperitoneally, or parenterally, by intravenous, intramuscular, topical, or subcutaneous routes, or by injection into tissue.

As used herein, “effective amount” or “therapeutically effective amount” is the amount of a composition of this disclosure which, when administered to a subject, is sufficient to effect treatment of a disease or condition in the subject. The amount of a composition of this disclosure which constitutes a “therapeutically effective amount” may vary depending on the composition, the condition and its severity, the manner of administration, and the age of the subject to be treated.

As used herein, “treating” or “treatment” means the treatment of a disease or condition of interest in a subject having the disease or condition of interest, and includes: (i) preventing the disease or condition from occurring in the subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, i.e., relieving pain without addressing the underlying disease or condition.

Still referring to FIG. 1, in an embodiment, augmented peptide composition 100 may include a therapeutic peptide moiety 104 conjugated to a polyethylene glycol (PEG) moiety, wherein the therapeutic peptide moiety 104 is identified as a function of integrated multiomic and immunophenotypic data associated with an individual using multiomic and immune modeling. A “moiety,” for purposes of this disclosure, is a functional group within a larger molecule. For example, a moiety may refer to a specific sub-structure or fragment of a compound that imparts particular chemical or biological properties, such as, without limitation benzene ring moiety, an amine moiety, a sugar moiety, and the like. For purposes of this disclosure, a “therapeutic peptide moiety” is the bioactive portion of an augmented peptide that confers a desired therapeutic effect. In an embodiment, the therapeutic peptide moiety 104 may include a segment that is specifically designed or selected, in some cases based on biological, multiomic, or immune modeling, to interact with specific cellular targets or pathways, thereby mediating treatment benefits. For example, the therapeutic peptide moiety 104 may be configured to improve longevity immunomodulation, treat weight aliments, and/or treat cancer depending on the targeted therapeutic effect.

With continued reference to FIG. 1, for purposes of this disclosure, “integrated multiomic data” refers to a dataset obtained by combining and analyzing multiple layers of biological information from the same samples or individuals. For example, multiple layers of biological information may include, but are not limited to genomics, transcriptomics, proteomics, metabolomics, epigenetics and the like. This integrative approach allows researchers to capture a holistic view of biological systems by linking genetic variations with changes in gene expression, protein abundance, metabolic profiles, and epigenetic modifications. The resulting dataset facilitates a deeper understanding of complex molecular interactions, regulatory networks, and disease mechanisms, ultimately enabling more precise diagnostics, personalized treatment strategies, and targeted therapeutic interventions. For purposes of this disclosure, “immunophenotypic data” refers to the detailed information gathered on immune cell populations by analyzing their characteristic markers. Immunophenotypic data may be obtained using techniques such as flow cytometry, mass cytometry (CyTOF), or immunohistochemistry, which allow for the simultaneous measurement of multiple cell surface or intracellular markers. The resulting data may provide insights into the identity, abundance, activation status, and functional state of various immune cell subsets. Such information may be crucial for understanding immune responses, diagnosing immunological disorders, monitoring disease progression, and developing targeted immunotherapies.

In further reference to FIG. 1, in an embodiment, identifying a therapeutic peptide moiety 104 using multiomic and immune modeling may involve integrating diverse layers of biological information, such as integrated multiomic and immunophenotypic data, to pinpoint key molecular targets and pathways that can be modulated for therapeutic benefit. For purposes of this disclosure, “multiomic modeling” refers to the integrated analysis of two or more “omics” data layers from the same biological sample or cohort. Further, for purposes of this disclosure, “immune modeling” described computational or mathematical frameworks that simulate, predict, or quantify aspects of the immune system's behavior. This dataset helps reveal aberrant signaling networks, gene expression patterns, protein modifications, and metabolic changes associated with a disease state. Following the reception of integrated multiomic data, immune modeling may be employed to profile the immune system's status and identify relevant immune cell populations, activation states, and cytokine profiles using techniques such as flow cytometry or mass cytometry. By integrating these immunophenotypic insights with the multiomic data, researchers can determine which molecular targets are not only dysregulated but also functionally relevant within the immune context of the disease. Once potential targets are identified, computational modeling and experimental validation may guide the design of peptide sequences that can interact with these targets. The therapeutic peptide moiety 104 may then be optimized to achieve high specificity and efficacy in modulating the target's activity, whether by mimicking endogenous regulatory peptides or inhibiting a pathogenic pathway. Throughout this process, considerations such as peptide stability, solubility, and potential immunogenicity may be addressed, often leading to further modifications like PEGylation or fusion with cell-penetrating domains. Ultimately, this integrated approach may ensure that the selected therapeutic peptide is tailored not only to the molecular abnormalities driving the disease but also to the intricate dynamics of the immune system, enhancing the potential for a targeted and effective treatment.

With continued reference to FIG. 1, in an embodiment, one or more aspects of identifying a therapeutic peptide moiety 104 may align with systems and methods described in attorney docket number 1399-001USC2USU1, U.S. patent application Ser. No. 18/396,506, filed on Dec. 26, 2023, titled “PRECISION-BASED IMMUNO-MOLECULAR AUGMENTATION (PBIMA) COMPUTERIZED SYSTEM, METHOD, AND THERAPEUTIC VACCINE,” which is incorporated by reference herein in its entirety.

In further reference to FIG. 1, for purposes of this disclosure, “conjugated” refers to the chemical process by which two or more distinct molecular entities are covalently bonded together to form a single compound. In an embodiment, the therapeutic peptide moiety 104 may be conjugated directly to the PEG moiety 108. Alternatively, the PEG moiety 108 may be first bonded to a linking agent 112, which may then serve as a bridge to attach to the therapeutic peptide moiety 104. This multi-step conjugation approach may provide enhanced control over the spacing, flexibility, and orientation of the components, ultimately improving the pharmacokinetic and pharmacodynamic properties of the final augmented peptide.

In continued reference to FIG. 1, in an embodiment, augmented peptide composition 100 may include a PEG moiety 108, such as PEG4. For purposes of this disclosure, “PEG” is a polyether compound composed of repeating ethylene oxide units. In an embodiment, adding a PEG moiety 108 may be referred to as PEGylation. For purposes of this disclosure, “PEGylation” is the process of covalently attaching PEG chains to another molecule. For example, PEGylation may include adding one or more PEG moieties to the therapeutic peptide, proteins, and/or other small molecules. Such a modification may enhance properties such as solubility, stability, and circulation half-life, while reducing immunogenicity and improving overall therapeutic efficacy. PEGylation may increase peptide half-life. PEGylation may also increase water solubility and reduce clearance through kidneys. An increased half-life may allow for sustained therapeutic effects and/or may reduce the need for frequent administration.

Still referring to FIG. 1, in an embodiment, augmented peptide composition 100 may include a linking agent 112, wherein the linking agent 112 is configured to link at least two molecular entities. For purposes of this disclosure, a “linking agent” is a chemical compound used to facilitate the attachment between two different molecular entities. In an embodiment, a linking agent 112 may act as a bridge, connecting two or more components while potentially providing spatial or functional benefits to the final conjugate. For example, a linking agent 112 may include an @-amino hexanoic acid (ahx) linker, a cell scaffold linkage, and a GCL linkage. In an embodiment, the PEG moiety 108 may be conjugated to the therapeutic peptide by one or more linking agents 112. In an embodiment, at least an adapter protein 116 may be conjugated to the therapeutic peptide moiety 104 by one or more linking agents 112. For purposes of this disclosure, an “adapter protein” is a non-enzymatic scaffold molecule that facilitates signal transduction by physically linking two or more other proteins into a functional complex. In an embodiment, linking agents 112 may be selected as a function of properties such as reactivity, stability, and compatibility with the molecules being linked, ensuring that the final product maintains the desired therapeutic properties.

With further reference to FIG. 1, in an embodiment, augmented peptide composition 100 may include a cell scaffold linkage, which may improve cellular permeability, intracellular delivery, and/or targeting precision. For purposes of this disclosure, a “cell scaffold linkage” is a chemical or biological bridging agent that covalently or non-covalently attaches a therapeutic moiety to a substrate designed to support cellular growth and organization. In an embodiment, this linkage may be engineered to be biocompatible and stable, ensuring that the therapeutic agent is effectively localized, delivered, or retained at the target site, thereby enhancing tissue integration, targeted delivery, or controlled release in biomedical applications.

In continued reference to FIG. 1, in an embodiment, augmented peptide composition 100 may include an ahx linker. For purposes of this disclosure an “ahx linker” is an @-amino acid with a hydrophobic, flexible structure. In some embodiments, replacing a section of a peptide backbone with a non-peptide component such as an ahx linker may reduce susceptibility of the molecule to proteolysis. In some embodiments, an ahx linker may provide chain flexibility to methylene bridges of a peptide without losing biological activity of the original form of the peptide. In some embodiments, inclusion of an ahx linker may improve peptide stability. For example, inclusion of an ahx linker may aid in resisting enzymatic degradation, ensuring structural integrity, and reducing the need for frequent re-administration.

In further reference to FIG. 1, in an embodiment, augmented peptide composition 100 may include a GCL linkage. For purposes of this disclosure, a “GCL linkage” refers to a γ-glutamyl-cysteine bond. In some embodiments, inclusion of a GCL linkage may increase permeability. Further, in an embodiment, inclusion of a GCL linkage may provide a stable connector between two molecular moieties, leveraging the inherent resistance of the γ-glutamyl-cysteine bond to proteolytic cleavage and its well-characterized biocompatibility.

With further reference to FIG. 1, in an embodiment, augmented peptide composition 100 may include at least an adapter protein 116, wherein the adapter protein 116 is selected as a function of a functional target. For purposes of this disclosure, a “functional target” is a desired therapeutic outcome. The goal of a functional target may include modulating a specific biological process rather than merely binding to a single molecular entity. In some embodiments, the adapter protein 116 is chosen based on its specific binding affinity for a molecular target, such as a cell surface receptor, biomarker, or other disease-associated molecule, that is implicated in the desired therapeutic outcome. This selection process can involve computational modeling, high-throughput screening, or other methodologies to identify adapter proteins 116 with optimal binding kinetics and specificity for the functional target. Additionally, the adapter protein 116 may include one or more binding domains or motifs that facilitate its interaction with the molecular target, thereby enhancing the targeted delivery or activity of the augmented peptide composition. In an embodiment, the at least an adapter protein 116 may include Growth Factor Receptor-Bound Protein 2 (GRB2), Src Homology 2 domain Containing (Shc), Nck, Crk, 14-3-3 Proteins, and/or the like. The inclusion of the adapter protein 116 thus may serve to further refine the therapeutic efficacy of the composition by ensuring that the conjugated therapeutic elements are effectively directed to their site of action.

In some embodiments, an augmented peptide may include a GGL domain. For purposes of this disclosure, a “GGL domain” is a domain found in the gamma subunit of the heterotrimeric G protein complex and in regulators of G protein signaling RGS proteins. The GGL domain may be characterized by a conserved structure that supports interactions with specific signaling partners, most notably by including a binding site for the GB5 subunit. The presence of this binding site may be critical for modulating G protein signaling, as it influences the assembly, stability, and regulatory functions of the protein complex. By incorporating a GGL domain, augmented peptide composition can leverage these regulatory properties to interact with GB5 or related signaling molecules, thereby affecting downstream signaling events. This design is particularly useful in therapeutic contexts where precise modulation of G protein-coupled signaling pathways is desired to achieve a specific clinical outcome.

With continued reference to FIG. 1, in an embodiment, the at least an adapter protein 116 may include a binding domain that specifically recognizes and binds a molecular target associated with a functional target. The binding domain may be engineered for high-affinity, high-specificity recognition of a molecular target that is causally linked to the desired therapeutic outcome, i.e. the functional target. In an embodiment, the binding domain may take the form of single-chain antibodies (scFv), nanobodies, designed ankyrin repeat proteins (DARPins), affibodies, or other scaffold proteins known to bind cell-surface receptors, disease-associated biomarkers, or soluble pathological ligands. Selection of the binding domain may be guided by its equilibrium dissociation constant (K_D), off-rate (k_off), and epitope specificity to ensure robust target engagement under physiological conditions. In some embodiments, the binding domain may be monovalent or multivalent to enhance avidity and receptor clustering. Linker sequences between the adapter scaffold and binding domain can be optimized for flexibility, protease resistance, and minimal immunogenicity. By specifically anchoring the therapeutic peptide moiety 104 to a molecular target implicated in the underlying disease process, the adapter protein 116 may localize the therapeutic payload to the site of action and initiate downstream modulation of the functional target pathway.

With further reference to FIG. 1, in some embodiments, an augmented peptide may include the domain of a cell penetrating peptide (CPP). For example, in some embodiments, an augmented peptide may include an arginylglycylaspartic acid (RGD) domain. Inclusion of an RGD domain may cause integrin-mediated endocytosis and/or endosomal escape. Inclusion of an RGD domain may aid an augmented peptide in efficiently entering a cell. In some embodiments, an augmented peptide may include a TAT cell penetrating peptide domain. Inclusion of a TAT CPP domain may improve an augmented peptide's ability to enter a cell and/or a nucleus. In an embodiment, the domain of a CPP may be attached to the therapeutic peptide moiety 104 at the N-terminal. Attaching a CPP domain to the N-terminal of the therapeutic peptide moiety 104 can have several effects on the overall composition and function of the augmented peptide. For one, positioning the CPP at the N-terminal may ensure that it is readily accessible, which can enhance its ability to interact with cellular membranes and facilitate uptake. This configuration can improve the peptide's solubility and stability, potentially influencing its pharmacokinetic profile and biodistribution. Additionally, the N-terminal attachment may affect the peptide's overall conformation, ensuring that the CPP does not interfere with the biological activity of the therapeutic moiety, while still efficiently promoting cellular or nuclear entry. This strategic placement ultimately allows the augmented peptide to achieve a balance between effective delivery and therapeutic action.

In continued reference to FIG. 1, in an embodiment, augmented peptide composition 100 may be administered in order to improve longevity immunomodulation, which may include improving longevity, providing for immune regulation, reducing inflammation, and/or reducing oxidative stress. Further, augmented peptide composition 100 may be administered to treat a weight ailment, such as weight loss, metabolism control, and/or appetite control. In some cases, augmented peptide composition may be administered as a cancer therapy.

In some embodiments, an effective amount of augmented peptide composition 100 selected from the list consisting of PEG-FGF21-FG, PEG-MOTS-C, PEG-KPV, PEG-PT-141, PEG-BPC-157, PEG-Thymosin-Beta-4, PEG-GHK-CU, PEG-Sclank, PEG-GHRP-6-HGH-FG, PEG-Thymulin, PEG-VIP, PEG-Thymosin-Alpha-1, PEG-Epithalon (Epitalon), PEG-Cerebro-FG (Enhanced Cerebrolysin), PEG-SS-31 (Elamipretide), PEG-Synapsin-FG, PEG-FOXO4-DRI-FG, PEG-LL-37 (Cathelicidin), PEG-Semax, PEG-Dihexa, PEG-Kisspeptin, PEG-Larazotide, PEG-KLOTHO-FG, PEG-Anti-Galcctin-3-PC, PEG-IL-10-FG, PEG_ARA-290, PEG-CJC-Ipamorelin, PEG-Semorelin, PEG-Tesamorelin, PEG_Met_Enkephalin, and PEG_TP-508 (Chrysalin) may be administered in order to improve longevity immunomodulation.

In some embodiments, an effective amount of augmented peptide composition 100 selected from the list consisting of PEG-Semaglutide, PEG-Tirzepatide, PEG-Retatrutide, PEG-Melanotan-2, and PEG-LEP-FG may be administered to treat a weight ailment.

In some embodiments, an effective amount of augmented peptide composition 100 selected from the list consisting of PEG-Lactoferrin-CPP-TPP, PEG-Defensin-Bcta-CPP-TPP, PEG-Magainin-2-TPP, PEG-Melittin-TPP, PEG-Cyclin-dependent Kinase Inhibitory Peptide (CKI), PEG-PNC-27-CPP-TPP, PEG-PNC-28-CPP-TPP, PEG-Nutlin-Peptide-CPP-TPP, PEG-Nutlin-Thymulin-Adjuvant-CPP-TPP, PEG-Bombesin-TPP, PEG-Somatostatin Potentiating Peptide (SPP), PEG-Kisspeptin-10-CPP-TPP (Metastin), and PEG-PEP27-CPP-TPP (Metastin) may be administered as a cancer therapy.

In some embodiments, augmented peptide composition 100 may be lyophilized after production and shipped in a lyophilized form, such as a powder. For purposes of this disclosure, “lyophilized” refers to a substance that has been processed through lyophilization. Lyophilization is commonly known as freeze-drying. The process of lyophilization may involve freezing augmented peptide composition 100 and then reducing the surrounding pressure to allow the frozen water to sublimate directly from the solid phase to the gas phase. The result may include a dry product that is more stable, has a longer shelf life, and is easier to store and transport compared to its liquid form. In an embodiment, augmented peptide composition 100 may be reconstituted with water and/or another product such as a pharmaceutically acceptable excipient. In some embodiments, lyophilized augmented peptides may be shipped in batches of 40, 60, or 120 samples.

In some embodiments, augmented peptide composition 100 may be formulated with a pharmaceutically acceptable excipient. Pharmaceutically acceptable excipients may include, for example, solvents, dispersion media, coatings, antibacterial and antifungal agents, isotonic and absorption delaying agents, liquid or solid fillers, diluents, excipients, manufacturing aids (such as lubricants, talc magnesium, calcium or zinc stearate, or steric acid), or solvent encapsulating materials. Each pharmaceutically acceptable excipient may be “acceptable” in the sense of being compatible with the other ingredients of the formulation and not injurious to the subject. Some examples of materials which may serve as pharmaceutically-acceptable excipients include, without limitation: (1) sugars, for example lactose, glucose, mannose and/or sucrose; (2) starches, for example corn starch and/or potato starch; (3) cellulose, and its derivatives, for example sodium carboxymethyl cellulose, methylcellulose, ethyl cellulose, microcrystalline cellulose and/or cellulose acetate; (4) powdered tragacanth; (5) malt; (6) gelatin; (7) lubricating agents, for example magnesium stearate, sodium lauryl sulfate and/or talc; (S) excipients, for example cocoa butter and/or suppository waxes; (9) oils, for example peanut oil, cottonseed oil, safflower oil, sesame oil, olive oil, corn oil and/or soybean oil; (10) glycols, for example propylene glycol; (11) polyols, for example glycerin, sorbitol, and/or mannitol; (12) esters, for example glycerides, ethyl oleate and/or ethyl laurate; (13) agar; (14) buffering agents, for example magnesium hydroxide and/or aluminum hydroxide; (15) alginic acid; (16) pyrogen-free water; (17) diluents, for example isotonic saline, and/or PEG400; (18) Ringer's solution; (19) C2-C12 alcohols, for example ethanol; (20) fatty acids; (21) pH buffered solutions; (22) bulking agents, for example polypeptides and/or amino acids (23) serum component, for example serum albumin, HDL and LDL; (24) surfactants, for example polysorbates (Tween 80) and/or poloxamers; and/or (25) other non-toxic compatible substances employed in pharmaceutical formulations: for example, fillers, binders, wetting agents, coloring agents, release agents, coating agents, sweetening agents, flavoring agents, perfuming agents, preservatives and/or antioxidants.

In some embodiments, a subject may be administered PEG-FGF21-FG. Fibroblast Growth Factor 21 (FGF21) is a protein crucial for metabolic regulation. It plays a pivotal role in glucose homeostasis, enhancing insulin sensitivity, promoting lipid metabolism, and inducing weight loss by increasing energy expenditure and encouraging “browning” of white adipose tissue. FGF21 is induced in response to stressors and has been implicated in cardiovascular health. Its functions extend to the central nervous system, and it may influence aging and longevity. Overall, FGF21 is a key player in maintaining metabolic balance and responding to various physiological challenges.

In some embodiments, a protocol for administration of PEG-FGF21-FG may be carried out with the following parameters: frequency: three times a week (3 IM injections); duration: 12 weeks with a 3-week break; dose: full dose is 1 ml vial; injectable: 5 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-MOTS-C. MOTS-C, a 16 amino acid mitochondrial derived peptide, is encoded from the 12S rRNA region of the mitochondrial genome. Under stress conditions, it is colocalized to mitochondria in various tissues and is found in plasma, but the levels decline with age. Since MOTS-C has important cellular functions as well as a possible hormonal role, it has been shown to have beneficial effects on age-related diseases including Diabetes, Cardiovascular diseases, Osteoporosis, postmenopausal obesity and Alzheimer's Disease. Aging is characterized by gradual loss of (mitochondrial) metabolic balance, decreased muscle homeostasis and eventual diminished physical capability, which potentially can be reversed with MOTS-C treatment.

In some embodiments, a protocol for administration of PEG-MOTS-C may be carried out with the following parameters: frequency: three times a week (3 IM injections); duration: 12 weeks with a 3-week break; dose: full dose is 1 ml vial; injectable: 5 mg/ml lyophilized powder provided in a 6 ml vial reconstitute to 1 ml (IM Injection); suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-KPV. KPV is a peptide that holds significance in the realm of cell communication and regulation. Comprising the amino acids Lysine (K), Proline (P), and Valine (V), this tripeptide has demonstrated potential effects on cellular functions. While its specific functions can vary, peptides like KPV are often studied for their ability to modulate cell signaling, influence protein-protein interactions, or participate in various physiological processes. As with many peptides, ongoing research aims to uncover the precise mechanisms and potential applications of KPV in areas such as cell biology and therapeutic development.

In some embodiments, a protocol for administration of PEG-KPV may be carried out with the following parameters: frequency: three times a week (3 IM injections); duration: 12 weeks with a 3-week break; dose: Full Dose is 1 ml vial; injectable: 2.5 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-PT-141 (Bremelanotide). PT-141, also known as Bremelanotide, is a synthetic peptide developed as a potential treatment for sexual dysfunction. It is designed to activate melanocortin receptors in the brain, particularly MC4R (melanocortin 4 receptor), which regulate sexual function, arousal, and desire. PT-141 has been studied for its ability to enhance libido and treat conditions such as female sexual arousal disorder (FSAD) and erectile dysfunction (ED). It is administered through subcutaneous injection and is thought to work by influencing the central nervous system to improve sexual response. As with any pharmaceutical compound, the use of PT-141 should be under the guidance of a qualified healthcare professional, and its safety and efficacy may vary for different individuals.

In some embodiments, a protocol for administration of PEG-PT-141 may be carried out with the following parameters: frequency: three times a week (3 IM injections); duration: 12 weeks with a 3-week break; dose: Full Dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-BPC-157. BPC-157, or Body Protection Compound-157, is a synthetic peptide derived from a natural protein segment in human gastric juice. Comprising 15 amino acids, BPC-157 has been studied for its potential therapeutic effects, including accelerated wound healing, promotion of tissue repair, angiogenesis, and anti-inflammatory properties. Research has focused on its applications in treating injuries to muscles and tendons and its potential benefits for gastrointestinal health. Typically administered through injection, BPC-157 shows promise in various medical contexts, but caution is advised, and consultation with healthcare professionals is essential due to potential variations in safety and efficacy.

In some embodiments, a protocol for administration of PEG-BPC-157 may be carried out with the following parameters: frequency: Three times a week (3 IM injections); duration: 12 weeks with a 3-week break; dose: full dose is 1 ml vial; injectable: 5 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Thymosin-Beta-4. Thymosin beta-4 (TB-4), a naturally occurring peptide, boasts remarkable potential in diverse therapeutic realms. Renowned for its proficiency in accelerating wound healing and tissue repair, TB-4 also showcases anti-inflammatory attributes, contributing to immune modulation. Studies have explored its cardioprotective effects, enhancing cardiac function and fostering angiogenesis—the creation of new blood vessels. With its pivotal role in cell migration and differentiation, TB-4 is a promising candidate for longevity benefits and immune system enhancement. While ongoing research continues to unravel its multifaceted applications, Thymosin beta-4 stands at the forefront of potential breakthroughs in wound healing, cardiovascular health, immune support, and overall tissue regeneration. As the landscape evolves, seek guidance from healthcare professionals for the latest insights and recommendations on TB-4 utilization.

In some embodiments, a protocol for administration of PEG-Thymosin-Beta-4 may be carried out with the following parameters: frequency: three times a week (3 IM injections); duration: 12 weeks with a 3-week break; dose: full dose is 1 ml vial; injectable: 5 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-GHK-CU by injection. GHK-Cu is a naturally occurring copper complex initially identified in human plasma and later found in various bodily fluids. With a high affinity for copper ions crucial for normal bodily functions, this peptide is pivotal in promoting wound healing, attracting immune cells, and exerting antioxidant effects. It stimulates collagen synthesis, exhibits anti-inflammatory properties, and supports blood vessel growth. As a feedback signal after tissue injury, GHK-Cu acts as a potent protector, controlling oxidative damage and facilitating tissue remodeling. Unfortunately, the decline in GHK-Cu concentration with age reduces anti-inflammatory and tissue regeneration effects, contributing to increased inflammation, cancerous activity, and tissue deterioration.

In some embodiments, a protocol for administration of PEG-GHK-CU may be carried out with the following parameters: frequency: three times a week (3 IM injections); duration: 12 weeks with a 3-week break; dose: full dose is 1 ml vial; injectable: 25 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-GHK-CU topically. GHK-Cu is a naturally occurring copper complex initially identified in human plasma and later found in various bodily fluids. With a high affinity for copper ions crucial for normal bodily functions, this peptide is pivotal in promoting wound healing, attracting immune cells, and exerting antioxidant effects. It stimulates collagen synthesis, exhibits anti-inflammatory properties, and supports blood vessel growth. As a feedback signal after tissue injury, GHK-Cu acts as a potent protector, controlling oxidative damage and facilitating tissue remodeling. Unfortunately, the decline in GHK-Cu concentration with age reduces anti-inflammatory and tissue regeneration effects, contributing to increased inflammation, cancerous activity, and tissue deterioration.

In some embodiments, a protocol for administration of PEG-GHK-CU may be carried out with the following parameters: frequency: three times a week (3 IM injections); duration: 12 weeks with a 3-week break; dose: full dose is 1 ml vial; content and potency: powder for 5% solution: 25 mg/ml lyophilized powder in a topical application, transdermal: 5 mg/ml (5%) topical foam provided in a 50 ml foaming applicator; suggested dosage: transdermal: apply 1 ml (2 pumps) to the scalp daily at night.

In some embodiments, a subject may be administered PEG-Selank. Selank, an ACTH/MSH-like peptide closely related to tufstin, is traditionally used for anxiety and depression, but its benefits extend to immune modulation, anticoagulation, PTSD, ADHD, and metabolic syndromes. With potent anxiolytic, neuropsychotropic, and antidepressant properties, Selank alleviates aggression and fear reactions without the side effects associated with benzodiazepine tranquilizers. It also acts as a nootropic, positively impacting memory and learning, and exhibits marked immunomodulatory activity. Clinical studies demonstrate its tranquilizing effects, while experiments reveal its potential in preventing weight gain, activating the anticoagulation system, and lowering blood glucose levels. Like Semax, Selank holds promise as a broad-spectrum therapeutic agent for treating metabolic syndrome.

In some embodiments, a protocol for administration of PEG-Selank may be carried out with the following parameters: frequency: three times a week (3 IM injections); duration: 12 weeks with a 3-week break; dose: Full Dose is 1 ml vial; injectable: 5 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-GHRP-6-HGH-FG (176-191). Members of the Growth Hormone-Releasing Peptide (GHRP) family stimulate growth hormone secretion and enhance the benefits of the growth hormone/insulin-like growth factor-1 (GH/IGF-1) axis. These peptides have demonstrated cardio, neuro, and broad cytoprotective properties. Examples include the regulation of cardiac electric potentials and ion pumps, promotion of myocardial trophism, support for cardiomyocyte survival, reduction of inflammatory soluble messengers and distal cellular effectors, mitigation of the impact of neurohormones in the myocardium, regulation of peripheral vascular tone, prevention of necrosis and apoptosis in various epithelial and non-epithelial structures, promotion of tissue remodeling by eliminating fibrotic material, stimulation of skeletal muscle trophism to control sarcopenia, reduction of cachexia and catabolismand overall cytoprotection, particularly before chemotherapy regimens, without associated proliferative stimulation.

In some embodiments, a protocol for administration of PEG-GHRP-6-HGH-FG (176-191) may be carried out with the following parameters: frequency: three times a week (3 IM injections); duration: 12 weeks with a 3-week break; dose: full dose is 1 ml vial; injectable: 5 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Thymulin. Thymulin, a pivotal biologically active peptide, plays a crucial role in immune function and exhibits notable neuroendocrine benefits. As a thymic hormone, it significantly contributes to the regulation of the immune system, influencing the maturation and functioning of T cells. Beyond its immune-modulating effects, Thymulin demonstrates potential neuroendocrine advantages, suggesting a broader impact on overall health. Research highlights its ability to regulate immune responses, making it a promising candidate for addressing immune deficiencies and promoting immune system balance. Ongoing investigations seek to unveil Thymulin's therapeutic applications, shedding light on its potential to influence both immune and neuroendocrine functions positively.

In some embodiments, a protocol for administration of PEG-Thymulin may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-VIP. VIP (Vasoactive Intestinal Peptide) is a neuropeptide known for its diverse physiological roles and potential therapeutic benefits. VIP influences various bodily functions as a neurotransmitter and immunomodulator, including vasodilation, immune system regulation, and anti-inflammatory responses. VIP has been studied for its neuroprotective effects and role in mitigating inflammation in neurodegenerative diseases. Additionally, VIP is implicated in promoting gastrointestinal health and modulating circadian rhythms. Research suggests that VIP may offer therapeutic avenues for conditions involving inflammation, immune dysregulation, and neurological disorders, positioning it as a promising peptide for further exploration in medical applications.

In some embodiments, a protocol for administration of PEG-VIP may be carried out with the following parameters: frequency: three times a week (3 IM injections); duration: 12 weeks with a 3-week break; dose: full dose is 1 ml vial; injectable: 5 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Thymosin-Alpha-1. Thymosin alpha-1, a peptide derived from the thymus gland, is vital in immune system regulation and response. Known for its immunomodulatory properties, Thymosin alpha-1 has been investigated for its potential therapeutic benefits in enhancing immune function. Studies suggest that it may boost the activity of various immune cells, such as T cells, and contribute to the defense against infections and diseases. Thymosin alpha-1 has shown promise in supporting individuals with weakened immune systems and those undergoing certain medical treatments. Its ability to modulate immune responses positions it as a potential adjunctive therapy for conditions where immune support is critical. Moreover, its application in personalized medicine is being explored, highlighting its potential to cater to individualized immune needs in diverse medical scenarios.

In some embodiments, a protocol for administration of PEG-Thymosin-Alpha-1 may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Epithalon (Epitalon). Epitalon, also known as Epithalon or Epithalone, is a synthetic peptide modeled after the naturally occurring Epithalamin. Produced in the brain's epithalamium-epiphyseal region, Epithalamin regulates metabolism, enhances hypothalamic sensitivity, normalizes pituitary function, and balances gonadotropins and melatonin levels. As a bio-regulator for the endocrine system, particularly the pineal gland, Epithalamin acts as an antioxidant, enhances stress resistance, and lengthens telomeres in human cells. Epitalon, replicating these functions, boasts complex mechanisms beyond telomerase activation, including reducing lipid oxidation, normalizing T cell function, regulating cholesterol, uric acid, and prolactin levels, and restoring pancreatic hormone function. Notably, it shows promise in normalizing melatonin levels in aging individuals, making it a multifaceted peptide with potential applications in promoting overall health and longevity.

In some embodiments, a protocol for administration of PEG-Epithalon may be carried out with the following parameters: frequency: three times a week (3 IM injections); duration: 12 weeks with a 3-week break; dose: full dose is 1 ml vial; injectable: 25 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Cerebro-FG (Enhanced Cerebrolysin). Cerebrolysin, a porcine-derived peptide preparation rich in free amino acids, boasts neuroprotective and neurotrophic properties. Featuring active peptide fragments like nerve growth factor, BDNF, Ciliary Nerve Growth Factor, P-21, enkephalins, and orexin, Cerebrolysin emulates the functions of endogenous neurotrophic factors crucial for neuron growth, maintenance, and repair. These properties extend to neuroprotection, strengthening neural pathways, and promoting synaptic plasticity. When administered as an analog to endogenous neurotrophic factors, Cerebrolysin proves effective in treating neurodegenerative diseases, offering both symptomatic relief and pathological suppression over the short and long term. Its neuroprotective attributes also hint at preventive potential for individuals with a genetic predisposition to Alzheimer's disease. Cerebrolysin's clinical significance is recognized in various regions, making it a valuable tool in addressing neurological challenges.

In some embodiments, a protocol for administration of PEG-Cerebro-FG may be carried out with the following parameters: frequency: three times a week (3 IM injections); duration: 12 weeks with a 3-week break; dose: full dose is 1 ml vial; injectable: 25 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-SS-31 (Elamipretide). SS-31, or Bendavia, is a mitochondrial-targeted peptide renowned for its potent antioxidant properties and the ability to penetrate mitochondria, which are crucial for cellular health and energy production. Focused on optimizing mitochondrial function, SS-31 exhibits promise in reducing oxidative stress, enhancing cellular respiration, and improving overall mitochondrial efficiency. Its potential anticancer and neuroprotective properties make it a compelling candidate for conditions linked to mitochondrial dysfunction. Moreover, ongoing research highlights SS-31's potential in personalized medicine, showcasing its adaptability for tailored therapeutic interventions based on individual health profiles. This positions SS-31 as a versatile peptide with multifaceted mitochondrial protection and health-promoting effects.

In some embodiments, a protocol for administration of PEG-SS-31 may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Synapsin-FG with (NAD+25 mg). Synapsin peptide emerges as a promising compound with potential benefits for cognitive health and neuroprotection. Synapsin aims to target synaptic function to enhance communication between neurons, supporting neurotransmitter release and synaptic plasticity. Research suggests that Synapsin may contribute to cognitive enhancement, improving memory, learning, and overall cognitive function. Additionally, its neuroprotective properties make it a candidate for addressing conditions related to synaptic dysfunction, such as neurodegenerative diseases. While further studies are needed to fully elucidate its mechanisms and clinical applications, Synapsin holds potential as a novel peptide with implications for cognitive well-being and neurological health.

In some embodiments, a protocol for administration of PEG-Synapsin-FG may be carried out with the following parameters: frequency: three times a week (3 IM injections); duration: 12 weeks with a 3-week break; dose: full dose is 1 ml vial; injectable: 5 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-FOXO4-DRI-FG. FOXO4-DRI peptide has garnered attention for its potential in senescence and age-related health. Specifically designed to target senescent cells, FOXO4-DRI aims to promote cellular clearance and mitigate the negative effects of cellular senescence. By disrupting the interaction between p53 and FOXO4 proteins, this peptide encourages the elimination of senescent cells, potentially contributing to tissue rejuvenation and age-related health improvements. Preliminary studies in animal models have shown promising results, suggesting that FOXO4-DRI may have implications for extending healthspan and addressing age-related conditions. However, as research is ongoing, further investigations are needed to understand its mechanisms and potential applications in human health comprehensively.

In some embodiments, a protocol for administration of PEG-FOXO4-DRI-FG may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-LL-37 (Cathelicidin). LL-37, a naturally occurring antimicrobial peptide derived from the cathelicidin protein, presents versatile benefits for immune defense, wound healing, and potentially longevity. Renowned for its potent antimicrobial properties, LL-37 aids in combating various pathogens and exhibits anti-inflammatory effects, modulating the immune response. Notably, LL-37 contributes to tissue repair and wound healing, making it a potential therapeutic agent for skin injuries. Emerging research also hints at its immunomodulatory effects, suggesting implications for autoimmune conditions. The peptide's multifunctionality, encompassing immune health, wound recovery, and potential longevity benefits, positions LL-37 as a promising avenue for further exploration in medical applications.

In some embodiments, a protocol for administration of PEG-LL-37 may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Semax. Semax peptide, a synthetic analog of adrenocorticotropic hormone (ACTH), has garnered attention for its potential cognitive and neuroprotective benefits. Recognized for its nootropic properties, Semax is believed to enhance cognitive functions, memory, and learning. It also exhibits potential neuroprotective effects, fostering neuronal survival against various stressors. Some studies suggest that Semax may have implications for longevity and healthspan, contributing to overall well-being. Notably, ongoing research continues to explore its diverse applications, making Semax a promising peptide for cognitive enhancement, neuroprotection, and potential contributions to longevity and extended healthspan.

In some embodiments, a protocol for administration of PEG-Semax may be carried out with the following parameters: frequency: three times a week (3 IM injections); duration: 12 weeks with a 3-week break; dose: full dose is 1 ml vial; injectable: 15 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Dihexa. Dihexa, a breakthrough peptide derived from angiotensin IV, stands at the forefront of neuroscientific innovation. Dihexa exhibits promising potential in neuroregeneration and cognitive enhancement research and is uniquely engineered for optimal blood-brain barrier penetration. This peptide is poised to redefine our understanding of neuroprotection and cognitive function, offering valuable insights into addressing neurodegenerative disorders and cognitive decline. In the dynamic landscape of peptide research, Dihexa emerges as a powerful tool, propelling the boundaries of neurological exploration and contributing to the evolution of cognitive science.

In some embodiments, a protocol for administration of PEG-Dihexa may be carried out with the following parameters: frequency: three times a week (3 IM injections); duration: 12 weeks with a 3-week break; dose: full dose is 1 ml vial; injectable: 5 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Kisspeptin. Kisspeptin peptide, initially acknowledged for its pivotal role in reproductive regulation, extends its influence on diverse physiological realms. Beyond its impact on fertility and reproductive hormone control, ongoing research suggests potential benefits in metabolic regulation and stress responses. Additionally, kisspeptin has shown promise in cancer research, where its regulatory influence on cellular proliferation and apoptosis points toward therapeutic applications. This multifaceted peptide's significance goes beyond its reproductive functions, positioning it as a valuable candidate for personalized medicine. With its unique properties, kisspeptin offers potential tailored interventions based on individual health profiles and conditions.

In some embodiments, a protocol for administration of PEG-Kisspeptin may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Larazotide. Larazotide acetate, a synthetic peptide known for its tight junction regulatory properties, holds promise in addressing intestinal permeability-related conditions, including celiac disease and certain gastrointestinal disorders. By enhancing barrier function in the intestines, Larazotide aims to mitigate the passage of undesirable substances into the bloodstream, potentially offering relief and improved quality of life. Additionally, its positive impact on gut health and inflammation control may contribute to its potential benefits in promoting longevity and overall well-being. While further research is needed to fully understand its mechanisms and long-term effects, Larazotide represents a peptide with multifaceted implications for digestive health and potential contributions to longevity.

In some embodiments, a protocol for administration of PEG-Larazotide may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-KLOTHO-FG. KLOTHO peptide, named after the Greek Fate who spins the thread of life, is associated with a myriad of potential health benefits. Originally identified for its anti-aging properties, KLOTHO is expressed in organs like the kidneys and brain, playing a crucial role in regulating various physiological processes. Research suggests that KLOTHO may have protective effects against age-related diseases, such as cardiovascular disease and neurodegenerative disorders. Moreover, it is implicated in promoting kidney health, regulating mineral metabolism, and enhancing cognitive function. KLOTHO's involvement in cellular processes and its potential to counteract age-related decline make it a compelling target for further exploration in the quest for improved longevity and overall health. Although the full extent of its benefits is still under investigation, KLOTHO peptide holds promise as a key player in the pursuit of healthier aging.

In some embodiments, a protocol for administration of PEG-KLOTHO-FG may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Anti-Galectin-3-PC. Galectin-3, a multifaceted protein implicated in immune responses and inflammatory processes, has been associated with various physiological functions and potential health benefits. Elevated levels of galectin-3 are linked to an increased risk of chronic inflammation, which is a contributing factor in conditions such as cardiovascular diseases, fibrosis, and autoimmune disorders. Mitigating this risk by using anti-galectin peptides holds promise in dampening the inflammatory response. Research suggests that interventions aimed at reducing galectin-3 activity may have potential therapeutic implications for managing inflammation-related diseases. By targeting galectin-3, anti-galectin peptides could play a role in modulating immune responses and mitigating inflammatory risk, offering a novel avenue for potential therapeutic interventions in conditions associated with aberrant immune activation and chronic inflammation.

In some embodiments, a protocol for administration of PEG-Anti-Galectin-3-PC may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-IL-10-FG. IL-10 fragment peptide, derived from the interleukin-10 (IL-10) cytokine, holds promise for its anti-inflammatory properties and potential therapeutic benefits. Interleukin-10 is known for its role in regulating immune responses and dampening inflammation. The IL-10 fragment peptide, designed to mimic the immunomodulatory effects of IL-10, may offer a targeted approach to mitigating excessive inflammatory responses. Research suggests that IL-10 and its fragments may be explored for their potential in managing inflammatory and autoimmune conditions. By modulating immune activity and promoting an anti-inflammatory environment, IL-10 fragment peptide could emerge as a valuable tool in the development of novel therapies for diseases characterized by dysregulated immune responses and chronic inflammation.

In some embodiments, a protocol for administration of PEG-IL-10-FG may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG_ARA-290 (Cibinetide). ARA-290, a peptide derived from erythropoietin, shows promise in various therapeutic applications. With anti-inflammatory properties, it has the potential to reduce inflammation in tissues, aiding in conditions characterized by excessive inflammation. Additionally, ARA-290 exhibits tissue repair and regenerative effects, making it valuable for healing damaged tissues. Some research suggests neuroprotective qualities, offering potential benefits for neurological conditions, while its analgesic properties may contribute to pain relief. Despite these promising aspects, ongoing research is essential to fully comprehend ARA-290's therapeutic potential and safety profile, emphasizing the importance of consultation with healthcare professionals for personalized advice.

In some embodiments, a protocol for administration of PEG_ARA-290 may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-CJC-Ipamorelin. CJC-1295 and Ipamorelin, when combined, form a potent peptide combination known for its potential benefits in enhancing growth hormone release and promoting overall well-being. CJC-1295 serves to extend the half-life of growth hormone-releasing hormone (GHRH), while Ipamorelin stimulates the release of growth hormone. Together, they synergistically amplify the pulsatile release of growth hormone, offering advantages such as improved muscle growth, enhanced fat metabolism, and potentially rejuvenated skin and connective tissues. This peptide combination may also contribute to increased energy levels, improved sleep quality, and a more robust immune system. While further research is ongoing, CJC-1295 and Ipamorelin have garnered attention for their potential in promoting physiological processes associated with growth hormone secretion, ultimately impacting overall health and vitality.

In some embodiments, a protocol for administration of PEG-CJC-Ipamorelin may be carried out with the following parameters: frequency: three times a week (3 IM injections); duration: 12 weeks with a 3-week break; dose: full dose is 1 ml vial; injectable: 6 mg/ml lyophilized powder provided in a 6 ml vial reconstitute to 1 ml administer IM; suggested dosage: inject 0.10 ml subcutaneously 5 out of 7 nights of the week before bedtime on an empty stomach. Continue the regimen clinically for 12 weeks, followed by a clinical pause of 3 weeks, before clinically initiating the next course with the full recommended dosage. Physician adjusted dosing is recommended.

In some embodiments, a subject may be administered PEG-Semorelin. Semorelin, a synthetic peptide known for stimulating growth hormone release, holds promise in promoting various health benefits, including increased lean muscle mass, improved metabolism, heightened energy levels, and potentially enhanced sleep quality. Its application extends to anti-aging strategies, where the modulation of growth hormone levels is often associated with the slowing of age-related decline. Moreover, Semorelin exemplifies the potential of personalized medicine, as its use can be tailored to individual health profiles for optimizing hormonal balance and overall well-being. Consulting with healthcare professionals is essential for a personalized approach to Semorelin therapy and monitoring its effects.

In some embodiments, a protocol for administration of PEG-Semorelin may be carried out with the following parameters: frequency: three times a week (3 IM injections); duration: 12 weeks with a 3-week break; dose: full dose is 1 ml vial; injectable: 3 mg/ml lyophilized powder provided in a 6 ml vial reconstitute to 1 ml administer IM; suggested dosage: inject 0.10 ml subcutaneously 5 out of 7 nights of the week before bedtime on an empty stomach. Continue the regimen clinically for 12 weeks, followed by a clinical pause of 3 weeks, before clinically initiating the next course with the full recommended dosage. Physician adjusted dosing is recommended.

In some embodiments, a subject may be administered PEG-Tesamorelin. Tesamorelin, a synthetic growth hormone-releasing hormone (GHRH) analogue, stands out for its potential in managing age-related concerns. By stimulating growth hormone release, Tesamorelin may contribute to reduced abdominal fat, increased lean muscle mass, and improved metabolic functions. Particularly effective in addressing visceral adiposity associated with aging, it offers a targeted approach to enhancing body composition. Tesamorelin's potential benefits extend to cognitive function, exercise capacity, and lipid profiles. As ongoing research explores its applications, Tesamorelin emerges as a focused solution for those seeking to optimize health amidst age-related hormonal changes. Its role in personalized medicine underscores its potential for tailored interventions based on individual health profiles.

In some embodiments, a protocol for administration of PEG-Tesamorelin may be carried out with the following parameters: frequency: three times a week (3 IM injections); duration: 12 weeks with a 3-week break; dose: full dose is 1 ml vial; injectable: 2.5 mg/ml lyophilized powder provided in a 6 ml vial reconstitute to 1 ml administer IM; suggested dosage: inject 0.5 ml subcutaneously before bed 6 out of 7 days 90 minutes after last food intake. Continue the regimen clinically for 12 weeks, followed by a clinical pause of 3 weeks, before clinically initiating the next course with the full recommended dosage. Physician adjusted dosing is recommended.

In some embodiments, a subject may be administered PEG_Met_Enkephalin. In some embodiments, a subject may be administered PEG_TP-508 (Chrysalin). In some embodiments, a subject may be administered PEG-Semaglutide. Semaglutide, a glucagon-like peptide-1 (GLP-1) receptor agonist, is widely recognized for its benefits in the management of type 2 diabetes. As a synthetic peptide, semaglutide mimics the actions of natural GLP-1, which helps regulate blood sugar levels by stimulating insulin release and reducing glucagon secretion. Beyond glycemic control, semaglutide has shown notable effects on body weight, making it a valuable option for individuals with obesity. Its once-weekly dosing regimen enhances patient adherence, and research suggests potential cardiovascular benefits. Moreover, semaglutide may have implications in personalized medicine for individuals with specific metabolic profiles. As a promising tool in the treatment landscape, semaglutide exemplifies the multifaceted impact of peptides in addressing metabolic disorders and improving overall health outcomes.

In some embodiments, a protocol for administration of PEG-Semaglutide may be carried out with the following parameters: frequency: three times a week (3 IM injections); duration: 12 weeks with a 3-week break; dose: full dose is 1 ml vial; injectable: 0.5 mg/ml lyophilized powder provided in a 6 ml vial reconstitute to 1 ml administer IM; suggested dosage: month 1:0.25 mg once a week, month 2:0.5 mg once a week, month 3:1 mg once a week. Physician adjusted dosing advised.

In some embodiments, a subject may be administered PEG-Tirzepatide. Tirzepatide, a groundbreaking dual glucose-dependent insulinotropic polypeptide (GIP) and glucagon-like peptide-1 (GLP-1) receptor agonist, presents a revolutionary approach to managing type 2 diabetes and obesity. Its unique dual action enhances insulin secretion, suppresses glucagon release, and induces satiety, resulting in superior glycemic control and substantial weight reduction. Clinical trials have highlighted its efficacy, surpassing existing therapies and showcasing cardiovascular benefits. Beyond these advantages, tirzepatide exhibits promise in promoting fat autophagy, offering a distinctive pathway to address obesity and metabolic disorders at the cellular level. With once-weekly dosing and multifaceted benefits, tirzepatide emerges as a transformative peptide, showcasing its potential as a comprehensive solution for individuals grappling with the complexities of type 2 diabetes, obesity, and metabolic health.

In some embodiments, a protocol for administration of PEG-Tirzepatide may be carried out with the following parameters: frequency: three times a week (3 IM injections); duration: 12 weeks with a 3-week break; dose: full dose is 1 ml vial; injectable: 2.5 mg/ml lyophilized powder provided in a 6 ml vial reconstitute to 1 ml administer IM; suggested dosage: month 1:2.5 mg once a week, month 2:5 mg once a week. Physician adjusted dosing advised

In some embodiments, a subject may be administered PEG-Retatrutide. Retatrutide, a single peptide with agonist activity at the glucose-dependent insulinotropic polypeptide (GIP), GLP-1, and glucagon receptors. This triagonist mode of action allows retatrutide to induce the glucose-dependent insulin release from the pancreas via GLP-1 receptors, enhance this GLP-1-mediated effect through GIP receptors, and increase energy expenditure through glucagon receptors. Retatrutide showed clinically meaningful glucose-lowering and bodyweight-lowering efficacy in a phase 1 study with no demonstrable toxicity.

In some embodiments, a protocol for administration of PEG-Retatrutide may be carried out with the following parameters: frequency: three times a week (3 IM injections); duration: 12 weeks with a 3-week break; dose: full dose is 1 ml vial; injectable: 2.5 mg/ml lyophilized powder provided in a 6 ml vial reconstitute to 1 ml administer IM; suggested dosage: consult physician for dosing schedule.

In some embodiments, a subject may be administered PEG-Melanotan-2. Melanotan-2, a synthetic peptide modeled after melanocortin, not only offers cosmetic benefits by inducing skin pigmentation but also demonstrates promising effects in various realms. Research indicates potential neuroprotective benefits, suggesting a role in supporting cognitive health. Users have reported heightened sexual performance as a noteworthy side effect, showcasing the peptide's impact on intimate well-being. Melanotan-2 further extends its influence to hormonal regulation, influencing key physiological processes. Additionally, ongoing investigations explore its potential in promoting longevity, marking it as a compound of interest with multifaceted benefits. As personalized medicine becomes integral in optimizing health, consultation with healthcare professionals is imperative to assess the suitability and safety of Melanotan-2 for individuals seeking benefits in neuroprotection, sexual performance, hormonal balance, and longevity.

In some embodiments, a protocol for administration of PEG-Melanotan-2 may be carried out with the following parameters: frequency: three times a week (3 IM injections); duration: 12 weeks with a 3-week break; dose: full dose is 1 ml vial; injectable: 10 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-LEP-FG. Leptin, recognized as the “satiety hormone,” plays a pivotal role in governing appetite, metabolism, and hormonal feedback. Recent research has delved into the potential benefits of specific Leptin Peptide Fragments, aiming to replicate or enhance leptin's regulatory actions. Envisioned advantages include robust support for weight management, precise regulation of appetite, and modulation of hormonal feedback mechanisms. This exploration instills confidence in the potential applications of Leptin Peptide Fragments in addressing conditions linked to leptin dysregulation.

In some embodiments, a protocol for administration of PEG-LEP-FG may be carried out with the following parameters: frequency: three times a week (3 IM injections); duration: 12 weeks with a 3-week break; dose: full dose is 1 ml vial; injectable: 5 mg/ml lyophilized powder provided in a 6 ml vial reconstitute to 1 ml administer IM; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Lactoferrin-CPP-TPP systemically and/or intratumorally. Lactoferrin peptide fragments, as synthetic precision peptides, exhibit notable potential in mitigating cancer. Derived from Lactoferrin, these fragments showcase efficacy in modulating the immune system, inhibiting angiogenesis, and regulating cell proliferation. The iron-binding capacity of Lactoferrin peptide fragments further contributes to limiting iron availability, a critical factor for cancer cell growth. Emerging evidence suggests that these synthetic peptides may not only demonstrate anticancer properties but also enhance the effectiveness of certain cancer treatments while potentially minimizing associated side effects. Ongoing research continues to unravel the precise mechanisms through which Lactoferrin peptide fragments exert their anticancer effects, offering promising avenues for cancer prevention and treatment strategies.

In some embodiments, a protocol for administration of PEG-Lactoferrin-CPP-TPP may be carried out with the following parameters: frequency: three times a week (3 IM injections); duration: 12 weeks with a 3-week break; dose: full dose is 1 ml vial; injectable: 1 mg/ml or greater of peptide mixture to administer IM/IV and intratumorally; suggested dosage: consult Intratumoral Injection Protocol, begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Defensin-Beta-CPP-TPP intratumorally. Defensin-Beta peptide, a naturally occurring antimicrobial peptide, has garnered interest for its potential benefits in cancer research. Studies suggest that Defensin-Beta may exhibit anticancer properties through its ability to selectively induce apoptosis, or programmed cell death, in cancer cells while sparing healthy cells. Additionally, Defensin-Beta peptide has demonstrated immunomodulatory effects, potentially enhancing the body's immune response against cancer cells. Its multifaceted action highlights its potential role in cancer therapeutics. While research is ongoing to further elucidate the mechanisms and clinical applications, Defensin-Beta peptide stands out as a promising candidate in the exploration of novel anticancer agents.

In some embodiments, a protocol for administration of PEG-Defensin-Beta-CPP-TPP may be carried out with the following parameters: frequency: three times a week (3 IM injections); duration: 12 weeks with a 3-week break; dose: full dose is 1 ml vial; injectable: 1 mg/ml or greater of peptide mixture to administer intratumorally; suggested dosage: consult Intratumoral Injection Protocol.

In some embodiments, a subject may be administered PEG-Magainin-2-TPP intratumorally. Magainin-2 peptide has garnered attention in cancer research due to its promising potential as an anticancer agent. This naturally occurring antimicrobial peptide exhibits multifaceted benefits in the context of cancer treatment. Magainin-2 has demonstrated the ability to selectively target and disrupt cancer cell membranes, inducing apoptosis, or programmed cell death, in malignant cells while sparing normal cells. Its antimicrobial properties also contribute to preventing infections, a common concern during cancer treatment. Moreover, Magainin-2's versatile nature allows it to modulate immune responses, enhancing the body's ability to recognize and eliminate cancer cells. The peptide's low toxicity and high specificity make it an appealing candidate for the development of novel and more effective cancer therapies, potentially offering a targeted and less invasive approach to treating various forms of cancer.

In some embodiments, a protocol for administration of PEG-Magainin-2-TPP may be carried out with the following parameters: frequency: three times a week (3 IM injections); duration: 12 weeks with a 3-week break; dose: full dose is 1 ml vial; injectable: 1 mg/ml or greater of peptide mixture to administer intratumorally; suggested dosage: consult Intratumoral Injection Protocol.

In some embodiments, a subject may be administered PEG-Melittin-TPP intratumorally. Melittin peptide, derived from bee venom, has shown promising potential in cancer therapy. One of its notable benefits lies in its ability to disrupt cancer cell membranes, leading to cell death through various mechanisms, including necrosis and apoptosis. Melittin exhibits selectivity toward cancer cells, minimizing harm to healthy cells. Additionally, its anti-inflammatory properties contribute to a reduction in the tumor microenvironment's pro-tumorigenic signals, hindering cancer progression. Melittin has demonstrated efficacy against a range of cancer types, suggesting its broad applicability. Furthermore, the peptide's relatively low toxicity and potential for targeted delivery make it an intriguing candidate for the development of innovative and more precise cancer treatments, offering a new avenue for therapeutic strategies in oncology.

In some embodiments, a protocol for administration of PEG-Melittin-TPP may be carried out with the following parameters: frequency: three times a week (3 IM injections); duration: 12 weeks with a 3-week break; dose: full dose is 1 ml vial; injectable: 1 mg/ml or greater of peptide mixture to administer intratumorally; suggested dosage: consult Intratumoral Injection Protocol.

In some embodiments, a subject may be administered PEG-Cyclin-dependent Kinase Inhibitory Peptide (CKI) systemically and/or intratumorally. Cyclin-dependent Kinase Inhibitory Peptide represents a promising advancement in cancer treatment. This peptide is designed to inhibit Cyclin-dependent Kinases (CDKs), crucial regulators of the cell cycle, thereby disrupting the uncontrolled proliferation of cancer cells. By specifically targeting CDKs, PEG-Cyclin-dependent Kinase Inhibitory Peptide can induce cell cycle arrest and promote apoptosis in cancer cells while sparing normal cells. The addition of polyethylene glycol (PEG) enhances the peptide's stability and prolongs its circulation in the bloodstream, allowing for sustained therapeutic effects. This targeted approach minimizes systemic toxicity and side effects associated with traditional chemotherapy. PEG-Cyclin-dependent Kinase Inhibitory Peptide holds significant promise in providing a more selective and efficacious treatment option for various cancer types, offering a potential breakthrough in precision medicine for cancer patients.

In some embodiments, a protocol for administration of PEG-Cyclin-dependent Kinase Inhibitory Peptide may be carried out with the following parameters: frequency: three times a week (3 IM injections); duration: 12 weeks with a 3-week break; dose: full dose is 1 ml vial; injectable: 1 mg/ml or greater of peptide mixture to administer IM/IV and intratumorally; suggested dosage: consult Intratumoral Injection Protocol, begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-PNC-27-CPP-TPP systemically and/or intratumorally. PNC-27 peptide has shown promising benefits in cancer treatment by targeting and disrupting cancer cell membranes. This synthetic peptide is designed to selectively bind to acidic cancer cell membranes, leading to membrane permeabilization and subsequent cell death through apoptosis. PNC-27's specificity for cancer cells minimizes harm to healthy cells, making it a potentially targeted and less toxic therapeutic option. Additionally, PNC-27 has demonstrated effectiveness against a variety of cancer types, suggesting its versatility in the realm of cancer treatment. Its ability to induce apoptosis in cancer cells without affecting normal cells highlights its potential as a novel and selective anticancer agent, providing a hopeful avenue for the development of innovative and targeted cancer therapies.

In some embodiments, a protocol for administration of PEG-PNC-27-CPP-TPP may be carried out with the following parameters: frequency: three times a week (3 IM injections); duration: 12 weeks with a 3-week break; dose: full dose is 1 ml vial; injectable: 1 mg/ml or greater of peptide mixture to administer IM/IV and intratumorally; suggested dosage: consult Intratumoral Injection Protocol, begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-PNC-28-CPP-TPP systemically and/or intratumorally. PNC-28 peptide has shown promising benefits in cancer treatment by targeting and disrupting cancer cell membranes. This synthetic peptide is designed to selectively bind to acidic cancer cell membranes, leading to membrane permeabilization and subsequent cell death through apoptosis. PNC-28's specificity for cancer cells minimizes harm to healthy cells, making it a potentially targeted and less toxic therapeutic option. Additionally, PNC-28 has demonstrated effectiveness against a variety of cancer types, suggesting its versatility in the realm of cancer treatment. Its ability to induce apoptosis in cancer cells without affecting normal cells highlights its potential as a novel and selective anticancer agent, providing a hopeful avenue for the development of innovative and targeted cancer therapies.

In some embodiments, a protocol for administration of PEG-PNC-28-CPP-TPP may be carried out with the following parameter: frequency: three times a week (3 IM injections); duration: 12 weeks with a 3-week break; dose: full dose is 1 ml vial; injectable: 1 mg/ml or greater of peptide mixture to administer IM/IV and intratumorally; suggested dosage: consult Intratumoral Injection Protocol, begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Nutlin-Peptide-CPP-TPP systemically and/or intratumorally. Nutlin-3 peptide fragment is a small peptide sequence that mimics the small molecule Nutlin-3. Specifically, that inhibits the interaction between p53 and MDM2, promoting the stabilization and activation of p53, a tumor suppressor protein. The p53 pathway plays a crucial role in preventing the development of cancer by regulating cell cycle progression and promoting apoptosis in response to DNA damage. By disrupting the interaction between p53 and MDM2, Nutlin-3 activates the p53 pathway, leading to the suppression of cancer cell growth and survival. Nutlin-3 and related compounds are being investigated for their potential therapeutic benefits in various cancers, particularly those with alterations in the p53 pathway. These compounds represent a novel approach in cancer treatment, aiming to restore the function of the p53 tumor suppressor protein to control and eliminate cancer cells.

In some embodiments, a protocol for administration of PEG-Nutlin-Peptide-CPP-TPP may be carried out with the following parameters: frequency: three times a week (3 IM injections); duration: 12 weeks with a 3-week break; dose: full dose is 1 ml vial; injectable: 1 mg/ml or greater of peptide mixture to administer IM/IV and intratumorally; suggested dosage: consult Intratumoral Injection Protocol, begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Nutlin-Thymulin-Adjuvant-CPP-TPP systemically and/or intratumorally. Innovative cancer treatments involve combining Nutlin-3, a peptide regulator targeting the p53 pathway, with immune modulators like thymulin to enhance natural defense mechanisms. This dual-action adjuvant strategy aims to suppress cancer cell growth by disrupting the p53-MDM2 interaction while boosting the immune response. This combination emerges as a promising form of adjunctive immune cancer treatment, offering a novel and potentially effective therapeutic avenue for cancer patients.

In some embodiments, a protocol for administration of PEG-Nutlin-Thymulin-Adjuvant-CPP-TPP may be carried out with the following parameters: frequency: three times a week (3 IM injections); duration: 12 weeks with a 3-week break; dose: full dose is 1 ml vial; injectable: 1 mg/ml or greater of peptide mixture to administer IM/IV and intratumorally; suggested dosage: consult Intratumoral Injection Protocol, begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-Bombesin-TPP intratumorally. Bombesin peptide has demonstrated potential benefits in cancer research, particularly in the realm of diagnostic imaging and targeted therapy. Due to its high affinity for bombesin receptors, which are overexpressed in various cancer types, bombesin peptide can be utilized as a molecular probe in imaging techniques such as positron emission tomography (PET). This enables the precise visualization and detection of tumors, aiding in early diagnosis and accurate staging. Moreover, bombesin peptide analogs have been explored for their therapeutic potential, acting as targeted agents to deliver anticancer drugs specifically to cancer cells that express bombesin receptors. This targeted approach holds promise for minimizing off-target effects and improving the efficacy of cancer treatments. While research is ongoing, bombesin peptide's unique characteristics make it a valuable tool in advancing both cancer diagnostics and targeted therapeutic interventions.

In some embodiments, a protocol for administration of PEG-Bombesin-TPP may be carried out with the following parameters: frequency: three times a week (3 IM injections); duration: 12 weeks with a 3-week break; dose: full dose is 1 ml vial; injectable: 1 mg/ml or greater of peptide mixture to administer intratumorally; suggested dosage: consult Intratumoral Injection Protocol.

In some embodiments, a subject may be administered PEG-Somatostatin Potentiating Peptide (SPP).

In some embodiments, a subject may be administered PEG-Kisspeptin-10-CPP-TPP (Metastin) systemically and/or intratumorally. Kisspeptin-10 peptide, a truncated form of the kisspeptin neuropeptide, has shown promising anticancer benefits. Research suggests that kisspeptin-10 may exhibit anti-proliferative effects on cancer cells, potentially influencing cellular growth and apoptosis. The peptide's ability to modulate key signaling pathways involved in cancer progression highlights its therapeutic potential. While further studies are necessary to comprehensively understand the mechanisms and clinical applications, kisspeptin-10 emerges as a candidate with anticancer properties, offering a potential avenue for the development of novel cancer therapies.

In some embodiments, a protocol for administration of PEG-Kisspeptin-10-CPP-TPP (Metastin) may be carried out with the following parameters: frequency: three times a week (3 IM injections); duration: 12 weeks with a 3-week break; dose: full dose is 1 ml vial; injectable: 1 mg/ml or greater of peptide mixture to administer IM/IV and intratumorally; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, a subject may be administered PEG-PEP27-CPP-TPP (Metastin) systemically and/or intratumorally. PEP27 peptide presents notable features as an anticancer strategy, benefiting from unique structural modifications that enhance its efficacy. Its increased hydrophobicity, particularly evident in the Pep27 plays a crucial role in penetrating the cell membrane and inducing apoptosis without triggering the release of cytochrome c from mitochondria. These distinctive characteristics, especially those demonstrated by Pep27, make PEP27 a compelling candidate for designing anticancer peptides. This highlights its potential to contribute to innovative and effective approaches in the field of cancer treatment.

In some embodiments, a protocol for administration of PEG-PEP27-CPP-TPP (Metastin) may be carried out with the following parameters: frequency: three times a week (3 IM injections); duration: 12 weeks with a 3-week break; dose: full dose is 1 ml vial; injectable: 1 mg/ml or greater of peptide mixture to administer IM/IV and intratumorally; suggested dosage: begin with an initial dose of 0.20 ml in week 1, considering patient tolerance and gradually escalate the dosage to 0.40 ml in week 2, 0.50 ml in week 3, and 0.75 ml in week 4. Reach the full prescribed dose of 1 ml in week 5, ensuring a measured and progressive adjustment based on the patient's response. Continue the regimen clinically for 12 weeks, followed by a clinical pause of 3 weeks, before clinically initiating the next course with the full recommended dosage.

In some embodiments, an augmented peptide described in Table 1 may be administered to a subject in order to improve longevity, provide for immune regulation, reduce inflammation, and/or reduce oxidative stress.

TABLE 1

| | | | Dosing / | Lead |
|---|---|---|---|---|
| Sequence # | Name | Sequence | mg | time |
| 1 | PEG_FGF-21_FG | PEG4-GGL-MDSDETGFE-ahx-PGILAPQP- | 5 | three |
| | sequence 1 | NH2 | | weeks |
| 2 | PEG_FGF-21_FG | PEG4-GGL-MDSDETGFE-ahx-PGKLAPQP-NH2 | 5 | three |
| | sequence 2 | | | weeks |
| 3 | PEG_MOTS-C | PEG4-GGL-MRWQEMGYIFYPRKLR-NH2 | 5 | two |
| | | | | weeks |
| 4 | PEG_KPV | PEG4-GGL-KPV-NH2 | 2.5 | two |
| | | | | weeks |
| 5 | PEG_PT-141 | PEG4-GGL-XDHFRWK-NH2 (Ac-Nle-Asp(1)- | 10 | three |
| | (Bremelanotide) | His-D-Phe-Arg-Trp-Lys(1)) | | weeks |
| | sequence 1 | | | |
| 6 | PEG_PT-141 | PEG4-GGL-{Nle}cyclo[Asp-His-D-Phe-Arg-Trp- | 10 | three |
| | (Bremelanotide) | Lys]-NH2 | | weeks |
| | sequence 2 | | | |
| 7 | PEG_BPC-157 sequence | PEG4-GGL-GEPPPGKPADDAGLV-NH2 | 5 | two |
| | 1 | | | weeks |
| 8 | PEG_BPC-157 sequence | PEG4-GGL-GEPKPGKPADDAGLV-NH2 | 5 | two |
| | 2 | | | weeks |
| 9 | PEG_Thymosin_Beta-4 | PEG4-GGL-[acetyl]-SDKPDMAEIEKFDKSKLK- | 5 | four |
| | sequence 1 | ahx-KTETQEKNPLPSKETIEQEKQAGES-NH2 | | weeks |
| 10 | PEG_Thymosin_Beta-4 | PEG4-GGL-SDKPDMAEIEKFDKSKLK-ahx- | 5 | four |
| | sequence 2 | KTETQEKNPLPSKETIEQEKQAGES-NH2 | | weeks |
| 11 | PEG_GHK-CU | PEG4-GGL-GHK-NH2.CU.xHAC | 25 | two |
| | Injectible | | | weeks |
| 12 | PEG_GHK-CU Topical | PEG4-GGL-GHK-ahx-ACSSSPSKHCG- | 25 | two |
| | | NH2.CU.xHAC | | weeks |
| 13 | PEG_Selank | PEG4-GGL-TKPRPGP-NH2 | 5 | two |
| | | | | weeks |
| 14 | PEG_GHRP- | PEG4-GGL-HWAWFK-ahx- | 10 | two |
| | 6_HGH_FG_176-191 | YLRIVQCRSVEGSCGF-NH2 | | weeks |
| | sequence 1 | | | |
| 14 | PEG_GHRP- | Disufide Bond required for stability, isomerization | 10 | two |
| | 6_HGH_FG_176-191 | and oxidation per BioPepTek Peptide Chem Team | | weeks |
| | sequence 2 | | | |
| 15 | PEG_Thymulin sequence | PEG4-GGL-XAKSQGGSN-NH2 | 10 | two |
| | 1 | | | weeks |
| 16 | PEG_Thymulin sequence | PEG4-GGL-K({pyr}AKSQGGSN)-NH2 | 10 | two |
| | 2 | | | weeks |
| 17 | PEG_VIP | PEG4-GGL- | 5 | three |
| | | HSDAVFTDNYTRLRKQMAVKKYLNSILN- | | weeks |
| | | NH2 | | |
| 18 | PEG_Thymosin_Alpha_ | PEG4-GGL-SDAAVDTSSEITT-ahx- | 10 | three |
| | 1 | KDLKEKKEVVEEAEN-NH2 | | weeks |
| 19 | PEG_Epithalon | PEG4-GGL-AEDG-NH2 | 25 | two |
| | (Epitalon) | | | weeks |
| 20 | PEG_Cerebro_FG | PEG4-GGL-DESHGTAVMWIFLKYP-NH2 | 25 | three |
| | | | | weeks |
| 21 | PEG_SS-31 | PEG4-GGL-RX-(2,6-diMe)-KF-NH2 | 10 | two |
| | (Elamipretide) sequence | | | weeks |
| | 1 | | | |
| 22 | PEG_SS-31 | PEG4-GGL-{D-R}-Tyr(2,6-diMe)-KF-NH2 | 10 | two |
| | (Elamipretide) sequence | | | weeks |
| | 1 | | | |
| 23 | PEG_Synapsin_FG with | PEG4-GGL-LRRRLSDANF-ahx-RRRRRRRR- | 5 | three |
| | (NAD+ 25 mg) | NH2 | | weeks |
| 24 | PEG_FOXO4-DRI_FG | PEG4-GGL--D-Leu-D-Thr-D-Leu-D-Arg-D-Lys- | 10 | three |
| | sequence 1 | D-Glu-D-Pro-D-Ala-D-Ser-D-Glu-D-Ile-D-Ala-D- | | weeks |
| | | Gln-D-Ser-D-Ile-D-Leu-D-Glu-D-Ala-D-Tyr-D- | | |
| | | Ser-D-Gln-D-Asn-D-Gly-D-Trp-D-Ala-D-Asn-D- | | |
| | | Arg-D-Arg-D-Ser-D-Gly-D-Gly-D-Lys-D-Arg-D- | | |
| | | Pro-D-Pro-D-Pro-D-Arg-D-Arg-D-Arg-D-Gln-D- | | |
| | | Arg-D-Arg-D-Lys-D-Lys-D-Arg-D-Gly-NH2 | | |
| 25 | PEG_FOXO4-DRI_FG | PEG4-GGL-D-Leu-D-Thr-D-Leu-D-Arg-D-Lys-D- | 10 | three |
| | sequence 2 | Glu-D-Pro-D-Ala-D-Ser-D-Glu-iso-Leu-D-Ala-D- | | weeks |
| | | Gln-D-Ser-iso-Leu-D-Leu-D-Glu-D-Ala-D-Tyr-D- | | |
| | | Ser-D-Gln-D-Asn-D-Gly-D-Trp-D-Ala-D-Asn-D- | | |
| | | Arg-D-Arg-D-Ser-D-Gly-D-Gly-D-Lys-D-Arg-D- | | |
| | | Pro-D-Lys-D-Pro-D-Arg-D-Arg-D-Arg-D-Gln-D- | | |
| | | Arg-D-Arg-D-Lys-D-Lys-D-Arg-D-Gly-NH2 | | |
| 26 | PEG_LL-37 | PEG4-GGL- | 10 | four |
| | (Cathelicidin) | LLGDFFRKSKEKIGKEFKRIVQRIKDFLRNLV | | weeks |
| | | PRTES-NH2 | | |
| 27 | PEG_Semax | PEG4-GGL-MEHFPGP-NH2 | 15 | three |
| | | | | weeks |
| 28 | PEG_Dihexa | PEG4-GGL- | 5 | three |
| | | THADILPTNVGSRGARAGLLGLKD-NH2 | | weeks |
| 29 | PEG_Kisspeptin | PEG4-GGL-YNWNSFGLRF-NH2 | 10 | three |
| | | | | weeks |
| 30 | PEG_Larazotide | PEG4-GGL-GGVLVQPG-NH2 | 10 | three |
| | | | | weeks |
| 31 | PEG_KLOTHO-FG | PEG4-GGL- | 10 | three |
| | | FQGTFPDGFLWAVGSAAYQTEGGWQQHGK | | weeks |
| | | G-NH2 | | |
| 32 | PEG_Anti-Galectin-3- | PEG4-GGL-IPGPTFLDPH-ahx-GPYTHDCPVK- | 10 | three |
| | PC | ahx-SGPIMLDEV-RGD-NH2 | | weeks |
| 33 | PEG_IL10-FG | PEG4-GGL-IQLLSSELLDE-NH2 | 10 | three |
| | | | | weeks |
| 34 | PEG_ARA-290 | PEG4-GGL-XEQLERALNSS-NH2 | 10 | |
| 35 | PEG_CJC_(1295)_ | PEG4-GGL- | 6 | two |
| | Ipamorelin | Y{d}ADAIFTQSYRKVLAQLSARKLLQDILSR- | | weeks |
| | | ahx-Aib-His-D-2Nal-D-Phe-Lys-NH2 | | |
| 36 | PEG_Semorelin | PEG4-GGL- | 3 | two |
| | | YADAIFTNSYRKVLGQLSARKLLQDIMSR- | | weeks |
| | | NH2 | | |
| 37 | PEG_Tesamorelin | PEG4-GGL-YADAIFTNSYRKVLGQLSARKL- | 2.5 | two |
| | | ahx-LQDIMSRQQGESNQERGARARL-NH2 | | weeks |
| 38 | PEG_Met_Enkephalin | PEG4-GGL-YGGFM-NH2 | 5 | |
| 39 | PEG_TP-508 (Chrysalin) | PEG4-GGL- | 5 | |
| | | AGYKPDEGKRGDACEGDSGGPFV-NH2 | | |

In some embodiments, an augmented peptide described in Table 2 may be administered to a subject in order to induce weight loss, control metabolism, and/or control hunger.

TABLE 2

| Sequence | | | Dosing / | |
|---|---|---|---|---|
| # | Name | Sequence | mg | Lead time |
| 40 | PEG_Semaglutide | PEG4-GGL- | 0.5 | four weeks |
| | sequence 1 | HAEGTFTSDVSSYLEGQAAK- | | |
| | | (PEG2-PEG2-γ-Glu-17- | | |
| | | carboxyheptadecanoyl)- | | |
| | | EFIAWLVRGRG-NH2 | | |
| 41 | PEG_Semaglutide | PEG4-GGL- | 0.5 | four weeks |
| | sequence 2 | H{Aib}EGTFTSDVSSYLEGQAAK- | | |
| | | (PEG2-PEG2-γ-Glu-17- | | |
| | | carboxyheptadecanoyl)- | | |
| | | EFIAWLVRGRG-NH2 | | |
| 42 | PEG_Tirzepatide | PEG4-GGL- | 2.5 | four weeks |
| | sequence 1 | YAGTFTSDYSILDKIAQEGE- | | |
| | | {diacid-γ-E-(AEEA)2-K}- | | |
| | | AFAQWLIAAGGPSGGPPPS-NH2 | | |
| 43 | PEG_Tirzepatide | PEG4-GGL- | 2.5 | four weeks |
| | sequence 2 | Y{Aib}EGTFTSDYSI{Aib}LDKIAQ- | | |
| | | {diacid-γ-E-(AEEA)2-K}- | | |
| | | AFVQWLIAGGPSSGAPPPS-NH2 | | |
| 44 | PEG_Retatrutide | PEG4-GGL- | 2.5 | four weeks |
| | sequence 1 | YA1QGTFTSDYSIL2LDKK4-ahx- | | |
| | | AQA1AFIEYLLEGGPSSGAPPPS3- | | |
| | | NH2 | | |
| 45 | PEG_Retatrutide | PEG4-GGL- | 2.5 | four weeks |
| | sequence 2 | Y{Aib}QGTFTSDYSK{αMe- | | |
| | | Leu } LDKK-(AEEA-γ-Glu-C20 | | |
| | | diacid)-ahx- | | |
| | | AQ{Aib}AFIEYLLEGGPSSGAPPPS- | | |
| | | NH2 | | |
| 46 | PEG_Melanotan | PEG4-GGL-Nle-Asp(1)-His-D-Phe- | 10 | three |
| | 2 | Arg-Trp-Lys(1)-NH2 | | weeks |
| 47 | PEG_LEP_FG | PEG4-GGL-WPYLFYVQ-ahx- | 5 | three |
| | sequence 1 | LQDMLWQLD-NH2 | | weeks |
| 48 | PEG_LEP_FG | PEG4-GGL-KPYLFYVQ-ahx- | 5 | three |
| | sequence 2 | LQDMLWQLD-NH2 | | weeks |

In some embodiments, an augmented peptide described in Table 3 may be administered to a subject as a cancer therapy.

TABLE 3

| Sequence | | | Dosing / | Lead |
|---|---|---|---|---|
| # | Name | Sequence | mg | time |
| 49 | PEG_Lactoferrin | PEG4-GRKKRRQRRR-ahx-RGD- | 1 | four |
| | CPP_TPP | MKLVFLVLLFLGAL-NH2 | | weeks |
| | (Systemic and | | | |
| | Intratumoral) | | | |
| | sequence 1 | | | |
| 50 | PEG_Lactoferrin_ | PEG4-GRKKRRQRRR-ahx-RGD- | 1 | four |
| | CPP_TPP | MKLKFLKLLKLGAL-NH2 | | weeks |
| | (Systemic and | | | |
| | Intratumoral) | | | |
| | sequence 2 | | | |
| 51 | PEG_Defensin- | PEG4-GGL-ACYCRIPACIAGERRY- | 1 | four |
| | Beta_CPP_TPP | RGD-GTCIYQGRLWAFCC-NH2 | | weeks |
| | (Intratumoral | | | |
| | Only) | | | |
| 52 | PEG_Magainin- | PEG4-GGL-GIGKFLHSAK-RGD- | 1 | three |
| | 2_TPP | KFGKAFVGEIMNS-NH2 | | weeks |
| | (Intratumoral | | | |
| | Only) | | | |
| 53 | PEG_Melittin_TPP | PEG4-GGL- | 1 | three |
| | (Intratumoral | GIGAVLKVLTTGLPALISWIKRKRQ- | | weeks |
| | Only) sequence 1 | RGD-NH2 | | |
| 54 | PEG_Melittin_TPP | PEG4-GGL- | 1 | three |
| | (Intratumoral | GIGAVLKVLTTGLPKLISWIKRKRQ- | | weeks |
| | Only) sequence 2 | RGD-NH2 | | |
| 55 | PEG_Cyclin- | PEG4-GRKKRRQRRR-ahx- | 1 | three |
| | dependent Kinase | TTYADFIASGRTGRRNAI-RGD-NH2 | | weeks |
| | Inhibitory Peptide | | | |
| | (CKI)(Systemic | | | |
| | and Intratumoral) | | | |
| 56 | PEG_PNC- | PEG4-GRKKRRQRRR-ahx- | 1 | three |
| | 27_CPP_TPP | PPLSQETFSDLWKLLOKKWKMRRN | | weeks |
| | (Systemic) | QFWVKVQRG-RGD-NH2 | | |
| | sequence 1 | | | |
| 57 | PEG_PNC- | PEG4-GRKKRRQRRR-ahx- | 1 | three |
| | 27_CPP_TPP | PPLSQETFSDLWKLLKKWKMRRNQ | | weeks |
| | (Systemic) | FWVKVQRG-RGD-NH2 | | |
| | sequence 2 | | | |
| 58 | PEG_PNC-28_ | PEG4-GRKKRRQRRR-ahx- | 1 | three |
| | CPP_TPP | ETFSDLWKLLOKKWKMRRNQFWV | | weeks |
| | (Systemic) | KVRG-RGD-NH2 | | |
| | sequence 1 | | | |
| 59 | PEG_PNC-28_ | PEG4-GRKKRRQRRR-ahx- | 1 | three |
| | CPP_TPP | ETFSDLWKLLKKWKMRRNQFWVK | | weeks |
| | (Systemic) | VQRG-RGD-NH2 | | |
| | sequence 2 | | | |
| 60 | PEG_Nutlin_Peptide_ | PEG4-GRKKRRQRRR-ahx- | 1 | three |
| | CPP_TPP | DWWPLAFEALLR-RGD-NH2 | | weeks |
| | (Systemic and | | | |
| | Intratumoral) | | | |
| 61 | PEG_Nutlin_ | PEG4-GGL-K(C5H5NO2- | 1 | three |
| | Thymulin_Comb_ | AKSQGGSN-OH)-RGD- | | weeks |
| | Adjuvant_CPP_TPP | DWWPLAFEALLR-NH2 | | |
| | (Systemic and | | | |
| | Intratumoral) | | | |
| 62 | PEG_Bombesin_TPP | PEG4-GRKKRRQRRR-ahx- | 1 | three |
| | (Intratumoral | QRLGHQWAVGHLM-RGD-NH2 | | weeks |
| | Only) sequence 1 | | | |
| 63 | PEG Bombesin_TPP | PEG4-GRKKRRQRRR-ahx- | 1 | three |
| | (Intratumoral | QRLGNQWAVGHLM-RGD-NH2 | | weeks |
| | Only) sequence 2 | | | |
| 64 | PEG-Somatostatin | PEG4-GGL-GRKKRRQRRR-ahx- | 1 | |
| | Potentiating | SANSNPAMAPRE-NH2 | | |
| | Peptide (SPP) | | | |
| 65 | PEG_Kisspeptin- | PEG4-GRKKRRQRRR-ahx- | 1 | three |
| | 10_CPP_TPP | YNWNSFGLRF-RGD-NH2 | | weeks |
| | (Metastin) | | | |
| | (Systemic and | | | |
| | Intratumoral) | | | |
| 66 | PEG_PEP27_CPP_ | PEG4-GRKKRRQRRR-ahx- | 1 | four |
| | TPP (For HER2 | MRKEFHNVLSSDQLLTDKRPARDY | | weeks |
| | Positive Cancer | NRK-RGD-NH2 | | |
| | Cells and Tumors) | | | |
| | (Systemic and | | | |
| | Intratumoral) | | | |

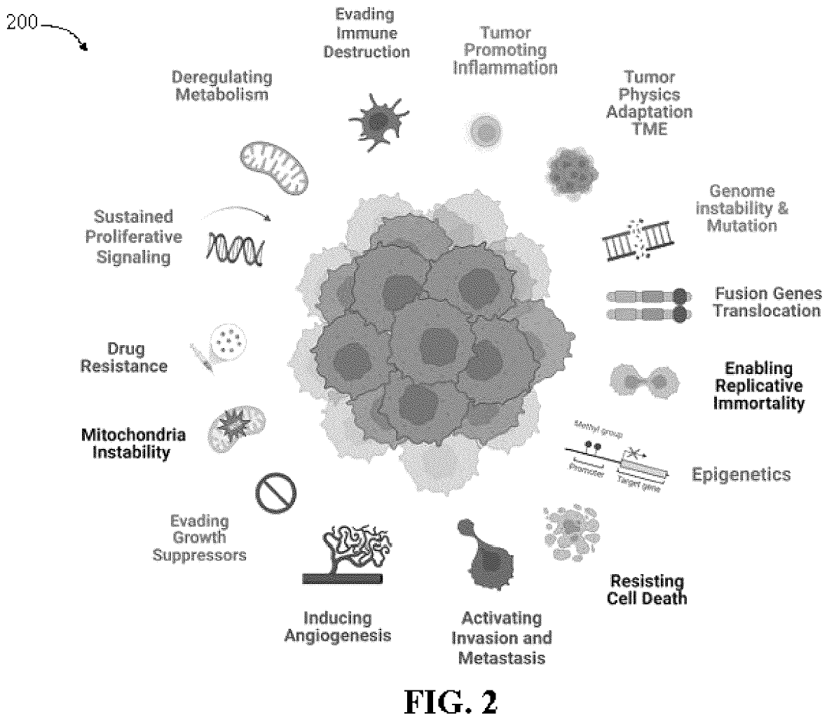

Referring now to FIG. 2, an illustration 200 describing hallmarks of cancer is provided. For purposes of this disclosure, “hallmarks” refer to the fundamental and defining characteristics or attributes that underlie a particular biological phenomenon or disease state. For example, in the field of oncology, the term “hallmarks of cancer” may be used to describe a set of distinctive features, such as sustained proliferative signaling, evasion of growth suppressors, resistance to cell death, enabling replicative immortality, induction of angiogenesis, and activation of invasion and metastasis, that collectively characterize the malignant phenotype. More generally, hallmarks may serve as key markers or criteria that can be used to identify, classify, or assess the state of a biological system or process.

In continued reference to FIG. 2, in some embodiments, an augmented peptide therapy described herein may counteract one or more mechanisms described in FIG. 2. For example, one or more augmented peptide compositions selected from a group consisting of augmented peptide composition may be selected from a group consisting of PEG-Lactoferrin-CPP-TPP, PEG-Defensin-Beta-CPP-TPP, PEG-Magainin-2-TPP, PEG-Melittin-TPP, PEG-Cyclin-dependent Kinase Inhibitory Peptide (CKI), PEG-PNC-27-CPP-TPP, PEG-PNC-28-CPP-TPP, PEG-Nutlin-Peptide-CPP-TPP, PEG-Nutlin-Thymulin-Adjuvant-CPP-TPP, PEG-Bombesin-TPP, PEG-Somatostatin Potentiating Peptide (SPP), PEG-Kisspeptin-10-CPP-TPP (Metastin), and PEG-PEP27-CPP-TPP (Metastin), to treat one or more aspects related to cancer.

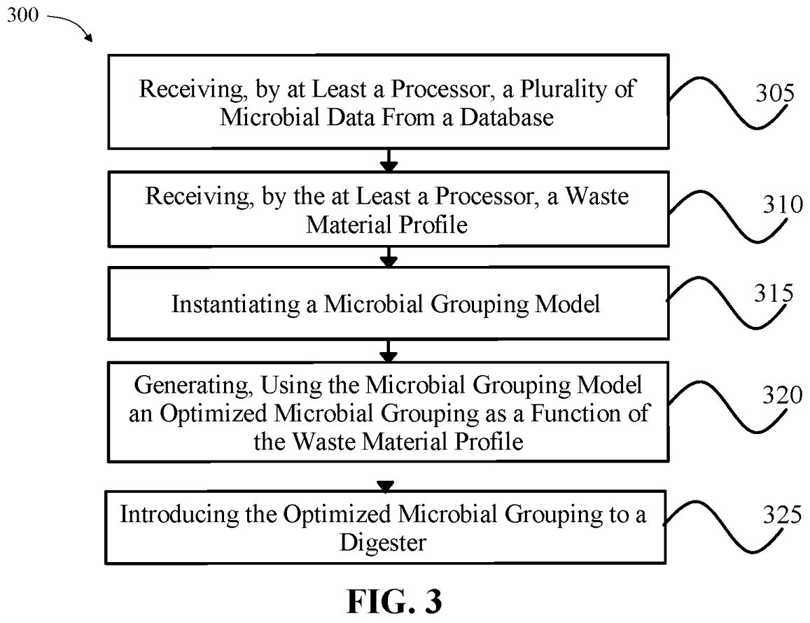

Now referring to FIG. 3, an exemplary method 300 for treating a disease or condition in a subject using an augmented peptide composition is illustrated. Method 300 may include a step 305 of receiving a plurality of molecular data associated with an individual. This may be implemented, without limitation, as referenced in FIGS. 1-2.

In continued reference to FIG. 3, method 300 may include a step 310 of identifying a therapeutic peptide moiety as a function of the plurality of integrated multiomic and immunophenotypic data using multiomic and immune modeling. This may be implemented, without limitation, as referenced in FIGS. 1-2.

With further reference to FIG. 3, method 300 may include a step 315 of conjugating a therapeutic peptide moiety to a PEG moiety to form an augmented peptide. This may be implemented, without limitation, as referenced in FIGS. 1-2.

Still referencing FIG. 3, method 300 may include a step 320 of conjugating at least a linker agent to the augmented peptide to link at least two molecular entities to form an augmented peptide composition. In an embodiment, the at least a linking against may include one or more of @-amino hexanoic acid (ahx) linker, a cell scaffold linkage, and a GCL linkage. This may be implemented, without limitation, as referenced in FIGS. 1-2.

In further reference to FIG. 3, method 300 may include a step 325 of administering an effective amount of the augmented peptide composition to the individual. In an embodiment, administering an effective amount of the augmented peptide composition may include intramuscular injection according to a regimen including three injections per week for a period of 12 weeks followed by a 3-week clinical pause. In an embodiment, the augmented peptide composition may be configured to improve longevity immunomodulation, wherein the augmented peptide composition is selected from a group consisting of PEG-FGF21-FG, PEG-MOTS-C, PEG-KPV, PEG-PT-141, PEG-BPC-157, PEG-Thymosin-Beta-4, PEG-GHK-CU, PEG-Sclank, PEG-GHRP-6-HGH-FG, PEG-Thymulin, PEG-VIP, PEG-Thymosin-Alpha-1, PEG-Epithalon (Epitalon), PEG-Cerebro-FG (Enhanced Cerebrolysin), PEG-SS-31 (Elamipretide), PEG-Synapsin-FG, PEG-FOXO4-DRI-FG, PEG-LL-37 (Catholicidin), PEG-Semax, PEG-Dihexa, PEG-Kisspeptin, PEG-Larazotide, PEG-KLOTHO-FG, PEG-Anti-Galectin-3-PC, PEG-IL-10-FG, PEG_ARA-290, PEG-CJC-Ipamorelin, PEG-Semorelin, PEG-Tesamorelin, PEG_Met_Enkephalin, and PEG_TP-508 (Chrysalin). Further, in an embodiment, the augmented peptide composition may be configured to treat a weight ailment, wherein the augmented peptide composition is selected from a group consisting of PEG-Semaglutide, PEG-Tirzepatide, PEG-Retatrutide, PEG-Melanotan-2, and PEG-LEP-FG. Further, in an embodiment, the augmented peptide composition may be configured to treat cancer, wherein the augmented peptide composition is selected from a group consisting of PEG-Lactoferrin-CPP-TPP, PEG-Defensin-Beta-CPP-TPP, PEG-Magainin-2-TPP, PEG-Melittin-TPP, PEG-Cyclin-dependent Kinase Inhibitory Peptide (CKI), PEG-PNC-27-CPP-TPP, PEG-PNC-28-CPP-TPP, PEG-Nutlin-Peptide-CPP-TPP, PEG-Nutlin-Thymulin-Adjuvant-CPP-TPP, PEG-Bombesin-TPP, PEG-Somatostatin Potentiating Peptide (SPP), PEG-Kisspeptin-10-CPP-TPP (Metastin), and PEG-PEP27-CPP-TPP (Metastin). This may be implemented, without limitation, as referenced in FIGS. 1-2.

With continued reference to FIG. 3, method 300 may further include a step of conjugating at least an adapter protein to the at least a linker agent. In an embodiment, the at least an adapter protein may include a binding domain that specifically recognizes and binds a molecular target associated with a functional target. This may be implemented, without limitation, as referenced in FIGS. 1-2.

In further reference to FIG. 3, in an embodiment, method 300 may further include a step of incorporating a cell penetrating peptide domain into the augmented peptide to enhance intracellular delivery. This may be implemented, without limitation, as referenced in FIGS. 1-2.

In continued reference to FIG. 3, method 300 may further include a step of providing the augmented peptide composition in a lyophilized form for reconstitution prior to administration. This may be implemented, without limitation, as referenced in FIGS. 1-2.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

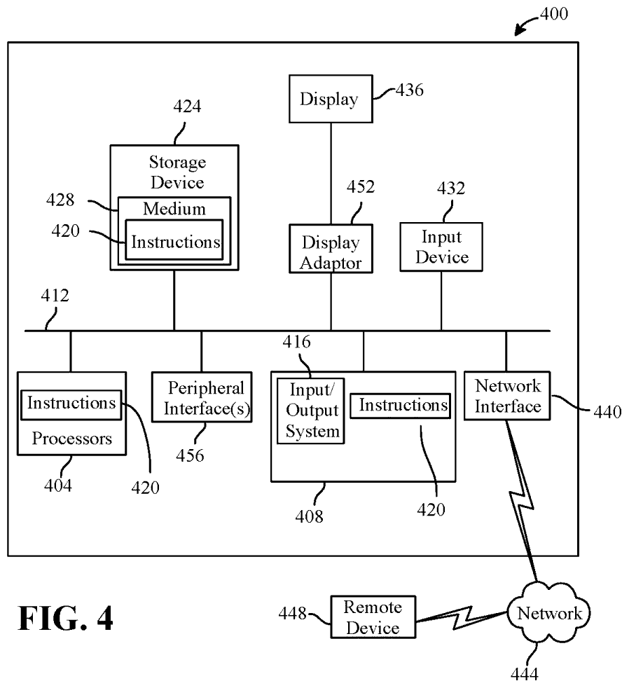

FIG. 4 shows a diagrammatic representation of one embodiment of a computing device in the exemplary form of a computer system 400 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 400 includes a processor 404 and a memory 408 that communicate with each other, and with other components, via a bus 412. Bus 412 may include any of several types of bus structures including, but not limited to, a memory bus, a memory controller, a peripheral bus, a local bus, and any combinations thereof, using any of a variety of bus architectures.

Processor 404 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 404 may be organized according to Von Neumann and/or Harvard architecture as a non-limiting example. Processor 404 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 408 may include various components (e.g., machine-readable media) including, but not limited to, a random-access memory component, a read only component, and any combinations thereof. In one example, a basic input/output system 416 (BIOS), including basic routines that help to transfer information between elements within computer system 400, such as during start-up, may be stored in memory 408. Memory 408 may also include (e.g., stored on one or more machine-readable media) instructions (e.g., software) 420 embodying any one or more of the aspects and/or methodologies of the present disclosure. In another example, memory 408 may further include any number of program modules including, but not limited to, an operating system, one or more application programs, other program modules, program data, and any combinations thereof.

Computer system 400 may also include a storage device 424. Examples of a storage device (e.g., storage device 424) include, but are not limited to, a hard disk drive, a magnetic disk drive, an optical disc drive in combination with an optical medium, a solid-state memory device, and any combinations thereof. Storage device 424 may be connected to bus 412 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 424 (or one or more components thereof) may be removably interfaced with computer system 400 (e.g., via an external port connector (not shown)). Particularly, storage device 424 and an associated machine-readable medium 428 may provide nonvolatile and/or volatile storage of machine-readable instructions, data structures, program modules, and/or other data for computer system 400. In one example, software 420 may reside, completely or partially, within machine-readable medium 428. In another example, software 420 may reside, completely or partially, within processor 404.

Computer system 400 may also include an input device 432. In one example, a user of computer system 400 may enter commands and/or other information into computer system 400 via input device 432. Examples of an input device 432 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 432 may be interfaced to bus 412 via any of a variety of interfaces (not shown) including, but not limited to, a serial interface, a parallel interface, a game port, a USB interface, a FIREWIRE interface, a direct interface to bus 412, and any combinations thereof. Input device 432 may include a touch screen interface that may be a part of or separate from display 436, discussed further below. Input device 432 may be utilized as a user selection device for selecting one or more graphical representations in a graphical interface as described above.

A user may also input commands and/or other information to computer system 400 via storage device 424 (e.g., a removable disk drive, a flash drive, etc.) and/or network interface device 440. A network interface device, such as network interface device 440, may be utilized for connecting computer system 400 to one or more of a variety of networks, such as network 444, and one or more remote devices 448 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 444, may employ a wired and/or a wireless mode of communication. In general, any network topology may be used. Information (e.g., data, software 420, etc.) may be communicated to and/or from computer system 400 via network interface device 440.

Computer system 400 may further include a video display adapter 452 for communicating a displayable image to a display device, such as display device 436. Examples of a display device include, but are not limited to, a liquid crystal display (LCD), a cathode ray tube (CRT), a plasma display, a light emitting diode (LED) display, and any combinations thereof. Display adapter 452 and display device 436 may be utilized in combination with processor 404 to provide graphical representations of aspects of the present disclosure. In addition to a display device, computer system 400 may include one or more other peripheral output devices including, but not limited to, an audio speaker, a printer, and any combinations thereof. Such peripheral output devices may be connected to bus 412 via a peripheral interface 456. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve compositions, methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. An augmented peptide composition, the augmented peptide composition comprising:
a therapeutic peptide moiety conjugated to a polyethylene glycol (PEG) moiety, wherein:
the therapeutic peptide moiety comprises a peptide moiety configured to treat an aspect of a cancer related ailment; and
the therapeutic peptide is selected from a group consisting of Lactoferrin-CPP-TPP, Defensin-Beta-CPP-TPP, Magainin-2-TPP, Melittin-TPP, Cyclin-dependent Kinase Inhibitory Peptide (CKI), PNC-27-CPP-TPP, PNC-28-CPP-TPP, Nutlin-Peptide-CPP-TPP, Nutlin-Thymulin-Adjuvant-CPP-TPP, Bombesin-TPP, Somatostatin Potentiating Peptide (SPP), Kisspeptin-10-CPP-TPP (Metastin), and PEP27-CPP-TPP (Metastin)
at least a linking agent, wherein the linking agent is configured to link at least two molecular entities.

2. An augmented peptide composition configured to treat an aspect of a cancer related ailment comprising:
a PEG moiety having four repeat units;
a cell-penetrating peptide segment comprising amino acid sequence GRKKRRQRRR;
a linker segment comprising 6-aminohexanioc acid (ahx);
a cell-adhesion peptide segment comprising amino acid sequence RGD; and
a hydrophobic peptide segment comprising amino acid sequence MKLVFLVLLFLGAL,
wherein the peptide is amidated at its C-terminus (—NH₂).

3. An augmented peptide composition configured to treat an aspect of a cancer related ailment comprising:
a PEG moiety having four repeat units;
a cell-penetrating peptide segment comprising amino acid sequence GRKKRRQRRR;
a linker segment comprising 6-aminohexanioc acid (ahx);
a cell-adhesion peptide segment comprising amino acid sequence RGD; and
a hydrophobic peptide segment comprising amino acid sequence MKLKFLKLLKLGAL,
wherein the peptide is amidated at its C-terminus (—NH₂).

4. An augmented peptide composition, the augmented peptide composition comprising:
a therapeutic peptide moiety conjugated to a PEG moiety, wherein the therapeutic peptide moiety is identified as a function of integrated multiomic and immunophenotypic data associated with an individual using multiomic and immune modeling; and
at least a linking agent, wherein the linking agent is configured to link at least two molecular entities.

5. The composition of claim 1, wherein the at least a linking agent comprises one or more of an ω-amino hexanoic acid (ahx) linker, a cell scaffold linkage, and a GCL linkage.

6. The composition of claim 1, wherein the therapeutic peptide moiety comprises a cell penetrating peptide domain to enhance intracellular delivery.

7. The composition of claim 6, wherein the cell penetrating peptide domain is attached to the therapeutic peptide moiety at an N-terminal.

8. The composition of claim 1, wherein:
the augmented peptide composition is configured to improve longevity immunomodulation; and
the augmented peptide composition is selected from a group consisting of PEG-FGF21-FG, PEG-MOTS-C, PEG-KPV, PEG-PT-141, PEG-BPC-157, PEG-Thymosin-Beta-4, PEG-GHK-CU, PEG-Selank, PEG-GHRP-6-HGH-FG, PEG-Thymulin, PEG-VIP, PEG-Thymosin-Alpha-1, PEG-Epithalon (Epitalon), PEG-Cerebro-FG (Enhanced Cerebrolysin), PEG-SS-31 (Elamipretide), PEG-Synapsin-FG, PEG-FOXO4-DRI-FG, PEG-LL-37 (Cathelicidin), PEG-Semax, PEG-Dihexa, PEG-Kisspeptin, PEG-Larazotide, PEG-KLOTHO-FG, PEG-Anti-Galectin-3-PC, PEG-IL-10-FG, PEG_ARA-290, PEG-CJC-Ipamorelin, PEG-Semorelin, PEG-Tesamorelin, PEG_Met_Enkephalin, and PEG_TP-508 (Chrysalin).

9. The composition of claim 1, wherein:
the augmented peptide composition is configured to treat a weight ailment; and
the augmented peptide composition is selected from a list consisting of PEG-Semaglutide, PEG-Tirzepatide, PEG-Retatrutide, PEG-Melanotan-2, and PEG-LEP-FG.

10. The composition of claim 1, wherein:
the augmented peptide composition is configured to treat cancer; and
the augmented peptide composition is selected from a group consisting of PEG-Lactoferrin-CPP-TPP, PEG-Defensin-Beta-CPP-TPP, PEG-Magainin-2-TPP, PEG-Melittin-TPP, PEG-Cyclin-dependent Kinase Inhibitory Peptide (CKI), PEG-PNC-27-CPP-TPP, PEG-PNC-28-CPP-TPP, PEG-Nutlin-Peptide-CPP-TPP, PEG-Nutlin-Thymulin-Adjuvant-CPP-TPP, PEG-Bombesin-TPP, PEG-Somatostatin Potentiating Peptide (SPP), PEG-Kisspeptin-10-CPP-TPP (Metastin), and PEG-PEP27-CPP-TPP (Metastin).

11. The composition of claim 1, wherein the augmented peptide is provided in a lyophilized form for reconstitution prior to administration.

12. A method of treating a disease or condition in a subject using an augmented peptide composition, the method comprising:
receiving integrated multiomic and immunophenotypic data associated with an individual;
identifying a therapeutic peptide moiety as a function of integrated multiomic and immunophenotypic data associated with an individual using multiomic and immune modeling;
conjugating the therapeutic peptide moiety to a PEG moiety to form an augmented peptide;
conjugating at least a linker agent to the augmented peptide to link at least two molecular entities; and
administering an effective amount of the augmented peptide composition to the individual.

13. The method of claim 12, wherein administering an effective amount of a composition comprising an augmented peptide comprises intramuscular injection according to a regimen comprising three injections per week for a period of 12 weeks followed by a 3-week clinical pause.

14. The method of claim 13, wherein the at least an adapter protein comprises a binding domain that specifically recognizes and binds a molecular target associated with a functional target.

15. The method of claim 12, wherein the at least a linking agent comprises one or more of an ω-amino hexanoic acid (ahx) linker, a cell scaffold linkage, and a GCL linkage.

16. The method of claim 12, further comprising incorporating a cell penetrating peptide domain into the therapeutic peptide moiety to enhance intracellular delivery.

17. The method of claim 12, wherein:
the augmented peptide composition is configured to improve longevity immunomodulation; and
the augmented peptide composition is selected from a group consisting of PEG-FGF21-FG, PEG-MOTS-C, PEG-KPV, PEG-PT-141, PEG-BPC-157, PEG-Thymosin-Beta-4, PEG-GHK-CU, PEG-Selank, PEG-GHRP-6-HGH-FG, PEG-Thymulin, PEG-VIP, PEG-Thymosin-Alpha-1, PEG-Epithalon (Epitalon), PEG-Cerebro-FG (Enhanced Cerebrolysin), PEG-SS-31 (Elamipretide), PEG-Synapsin-FG, PEG-FOXO4-DRI-FG, PEG-LL-37 (Cathelicidin), PEG-Semax, PEG-Dihexa, PEG-Kisspeptin, PEG-Larazotide, PEG-KLOTHO-FG, PEG-Anti-Galectin-3-PC, PEG-IL-10-FG, PEG_ARA-290, PEG-CJC-Ipamorelin, PEG-Semorelin, PEG-Tesamorelin, PEG_Met_Enkephalin, and PEG_TP-508 (Chrysalin).

18. The method of claim 12, wherein:
the augmented peptide composition is configured to treat a weight ailment; and
the augmented peptide composition is selected from a list consisting of PEG-Semaglutide, PEG-Tirzepatide, PEG-Retatrutide, PEG-Melanotan-2, and PEG-LEP-FG.

19. The method of claim 12, wherein:
the augmented peptide composition is configured to treat cancer; and
the augmented peptide composition is selected from a group consisting of PEG-Lactoferrin-CPP-TPP, PEG-Defensin-Beta-CPP-TPP, PEG-Magainin-2-TPP, PEG-Melittin-TPP, PEG-Cyclin-dependent Kinase Inhibitory Peptide (CKI), PEG-PNC-27-CPP-TPP, PEG-PNC-28-CPP-TPP, PEG-Nutlin-Peptide-CPP-TPP, PEG-Nutlin-Thymulin-Adjuvant-CPP-TPP, PEG-Bombesin-TPP, PEG-Somatostatin Potentiating Peptide (SPP), PEG-Kisspeptin-10-CPP-TPP (Metastin), and PEG-PEP27-CPP-TPP (Metastin).

20. The method of claim 12, further comprising providing the augmented peptide composition in a lyophilized form for reconstitution prior to administration.
